(19) United States
(12) Patent Application Publication
(10) Pub. No.: US 2026/0284225 A1
(43) Pub. Date: Sep. 24, 2026

(54) DELIVERY OF NUCLEIC ACIDS USING A TARGETED PEPTIDE CARRIER SYSTEM

(71) Applicant: Purdue Research Foundation, West Lafayette, IN (US)

(72) Inventors: David Thompson, West Lafayette, IN (US); Aayush Aayush, West Lafayette, IN (US); Saloni Darji, West Lafayette, IN (US)

(73) Assignee: Purdue Research Foundation, West Lafayette, IN (US)

(21) Appl. No.: 19/475,191

(22) PCT Filed: Apr. 19, 2024
(86) PCT No.: PCT/US2024/025515

§ 371 Date: Oct. 14, 2025

Related U.S. Application Data
(60) Provisional application No. 63/460,818, filed on Apr. 20, 2023.

(51) Int. Cl. A61K 48/00; A61K 31/7105; A61K 47/61; A61K 47/62; A61P 35/00

(52) U.S. Cl. CPC A61K 48/0041; A61K 31/7105; A61K 47/61; A61K 47/62; A61P 35/00

(57) ABSTRACT

A layer-by-layer ELP-nucleic acid (NA) nanoparticle (LENN) complex for targeted delivery of a therapeutic NA cargo to tumor cells; a method of formulating a LENN complex using a layer-by-layer deposition process (LbL); LENN complex formulations; pharmaceutical compositions comprising a LENN complex formulations; and methods of treating bladder cancer in a subject via LENN-mediated delivery of a therapeutic NA cargo.

### TECHNICAL FIELD

The present disclosure generally relates to a composition of matter and a method for cancer treatment. In particular, elastin-like polypeptide (ELP) carrier systems are disclosed for use in delivery of a nucleic acid cargo to tumor cells.

### BACKGROUND

Unraveling the human genome has opened many avenues to control and utilize genetic information to fight a vast array of diseases. Based upon this genetic information and driver mutations, nucleic acid (NA)-based therapies comprising siRNA, miRNA, mRNA, dsDNA, and other NA constructs are being explored to expand the druggable sites of the genome. Realizing the vast potential of these approaches requires that the NA be able cross multiple physiological and cellular barriers to reach its intended site of action. Unfortunately, the high negative charge density, hydrophilicity, and rapid clearance of unmodified NA from circulation make intracellular access difficult. Lessons from viruses regarding the storage and transfer of genetic information have guided researchers toward designing viral and non-viral delivery systems that deliver the NA cargo to the cytoplasm of target cells using some of their design principles.

Viruses and virus-like particles encapsulate their NA cargo to prevent degradation and pathogen-associated molecular pattern recognition, and to deliver the NA cargo to the host cell cytoplasm. Virus encapsulated NA cargo often displays a tropism for specific cell types that reduces off-target effects. Since these carriers have evolved to package a specific genome size, they have cargo size limits that challenge their adaptable manufacture, often require surface residue engineering to escape immune surveillance, and can be challenging to reliably manufacture on a large scale. Lipid-based nanoparticle (LNP) delivery systems were developed to address some of the limitations of the virus-encapsulated carriers. The most successful formulations utilize ionizable cationic lipids to promote the endosomal release of the NA cargo, forming the basis of Alnylam's Patisiran and the coronavirus vaccines developed by Bio-N-Tech/Pfizer and Moderna. Recent data shows that N-oxidation of the ionizable lipid during manufacturing and/or storage leads to byproducts that react to form lipidated mRNA, rendering it untranslatable. This explains, in part, the low biological efficiency of LNP on a NA copy number basis, since LNPs only deliver about 1% of the endocytosed NA cargo into the cytoplasm. Cationic polymers are another large class of biological and synthetic materials that control transfection complexes' functionality and biophysical properties. Polyethyleneimine (PEI) is one of the commonly used polymeric transfection agents, but even PEI has a very low ratio of nuclear plasmid per cell to total plasmid administered per cell—at about 0.8%.

Peptide-based formulations generally have the advantage of facile incorporation of targeting ligands via solid-phase peptide synthesis or recombinant protein approaches. Elastin-like polypeptides (ELP) have emerged as a promising class of non-immunogenic and biocompatible macromolecules that can be endowed with tunable stimuli-sensitive properties for various biomedical applications ranging from tissue engineering to gene delivery. Comprised of a Val-Pro-Gly-Xaa-Gly, or VPGXG, sequence (where X is any amino acid except proline) repeated multiple times, ELP typically have very high hydropathies. Recent work from Purdue University has utilized this property for rapid purification using an organic solvent extraction/precipitation workflow with retention of biological activity and removal of host cell NA and lipopolysaccharides from ELP fusions expressed in E. coli. (see Sweet et al., Biomacromolecules 22(5): 1990-1998 (2021), which is incorporated herein by reference).

Disease in the bladder is difficult to treat since the instillation of chemo- or immunotherapies into the bladder is confounded by the constant urine influx and voiding that limits target engagement times, typically less than 2 hours for drugs delivered via intravesical instillation. While these therapies could be delivered through other modes, they generally reduce tumor access and increase side effects. For extended contact with the bladder along with improved tumor selectivity, both small molecule and macromolecular drugs have been shown to benefit from targeting approaches. Systemic chemo- or immunotherapy delivery is another alternative typically reserved for about 20% of patients diagnosed with late-stage, muscle-invasive bladder cancer. Due to the high recurrence rate of bladder cancer, complete remission generally requires a combination of surgery and adjuvant chemo- and/or immunotherapy. The most common course of action is the instillation of live attenuated Bacillus Calmette Guerin (BCG) into the bladder after surgical resection of the detectable tumor. The co-localization of BCG mycobacteria with tumor tissue, enabled by the bacterial adhesion known as fibronectin attachment protein, leads to the immunogenic elimination of tumors near the mycobacterial adhesion site. Although this therapy has been a substantial advancement in bladder cancer immunotherapy, over 30% of patients are still unresponsive to this treatment modality. Additionally, because BCG is a live mycobacterium, there is some risk of systemic infection if the organism escapes the bladder compartment. A further complication of BCG therapy is a history of batch-to-batch variability, leading to brittle availability due to quality issues that negatively impact the BCG supply chain.

### SUMMARY

The present technology relates to highly-tunable elastin-like polypeptide (ELP) carrier systems for targeted delivery of a nucleic acid cargo to tumor cells In particular aspects, the technology disclosed herein includes (1) a layer-by-layer ELP-nucleic acid (NA) nanoparticle (LENN) for targeted, intracellular delivery of an NA therapeutic, (3) methods of formulated a LENN complex using a layer-by-layer deposition process (LbL), and (4) a method treating bladder cancer via LENN-mediated delivery of a therapeutic NA cargo.

In one aspect, a method of preparing an elastin-like polypeptide (ELP) carrier system for delivery of a nucleic acid cargo to overexpressing epidermal growth factor receptor (EFGR) tumor cells is provided, including the steps of: complexing nucleic acid material of the nucleic acid cargo with decaarginine-β-cyclodextrin (CD) to yield a CD-BP polyplex cargo; and coating the CD-BP polyplex cargo with an ELP-epidermal growth factor (ELP-EGF) fusion protein to yield the ELP carrier system. By way of example, the condensed NA material can be one or more of: siRNA, pDNA, miRNA, mRNA, and dsDNA. In the same or other examples, the tumor cells can be bladder tumor cells. In the same or yet other examples. ELP is a (VPGXG) n construct, wherein n represents the number of pentapeptide repeats and X represents a predetermined amino acid. For instance, X can be valine and n=24.

In another aspect, an elastin-like polypeptide (ELP) carrier system for delivery of a nucleic acid cargo to over-expressing EGFR (epidermal growth factor receptor) tumor cells is provided. In examples, the ELP carrier system includes (1) a nucleic acid material complexed with decaarginine-β-cyclodextrin (CD); and (2) an outer coating wrapping, at least in part, the CD-complexed nucleic acid material with one or more ELP-epidermal growth factor (ELP-EGF) fusion proteins. In examples, the nucleic acid material can comprise one or more of: siRNA, pDNA, lncRNA, miRNA, mRNA, and dsDNA. In the same or other examples, the tumor cells can be bladder tumor cells. In the same or yet other examples, the ELP is a (VPGXG), construct, wherein n represents the number of pentapeptide repeats and X represents a predetermined amino acid. For instance, X can be valine and n=24.

In certain embodiments, the ELP carrier system can be a layer-by-layer ELP-nucleic acid (NA) nanoparticle (LENN) complex, according to which the LENN complex can include a polyion complex core wrapped, at least in part, by an ELP fusion protein coating. In examples, the polyion complex core includes a nucleic acid cargo condensed by an oligocation and the ELP fusion protein comprises an EFP linked directly or indirectly to a co-expressed targeting ligand. In the same or still other examples, the oligocation is decaarginine-β-cyclodextrin (CDPLR10) or 2.5 kD polyethyleneimine-modified β-cyclodextrin (CDPEI_{2.5k}).

The nucleic acid cargo according to examples can be a therapeutic DNA, which can include one or more of pDNA, mcDNA, linear DNA amplicons, ASOs, CpG oligodeoxynucleotides, DNA enzymes, and DNA methyltransferase inhibitors. The NA cargo can also be a therapeutic RNA, which can include one or more of mRNA, siRNA, shRNA, miRNA, saRNA, piRNA, eRNA, lncRNA, circRNA, a ribozyme, an antagonist RNA aptamer, and a cell type-specific RNA aptamer. The NA cargo can also be a CRSPR-Cas9 vector.

In embodiments in which the LENN complex is used to treat bladder tumor cells, the targeting ligand can be a peptide or protein capable of selective interaction with a targeted membrane protein (or glycoprotein) implicated in cancer behavior, the expression of which can be characterized as either (1) overexpressed (or upregulated) in bladder tumors compared to normal tissues or (2) aberrantly expressed in bladder tumors but not in normal tissues. By way of example, the targeting ligand can be a peptide or protein capable of selective interaction with an upregulated integrin, receptor tyrosine kinase (RTK), or G-protein coupled receptor. For instance, the targeting ligand can be a peptide or protein capable of selective interaction with one of αEβ7 integrin, integrin α2β1, integrin β8, EGFR, FGFR, HER 2, VEGFR-2, or chemokine receptor 7 (CCR7). In one example, in which the membrane protein is EGFR, the ELP fusion protein can be N24EGF.

By way of further example, the targeting ligand can be a peptide or protein capable of selective interaction with an aberrantly expressed cell surface adhesion molecules (CAMs). For instance, the targeting ligand can be a peptide or protein capable of selective interaction with either NECTIN-4 or the surface adhesion glycoprotein TROP-2.

In certain embodiments, a method of formulating a LENN complex using a layer-by-layer deposition process (LbL) is provided, in which the method includes the steps of (1) condensing a nucleic acid material with a β-cyclodextrin oligocation to form a polyion complex; and (2) wrapping at least a portion of the polyion complex with an ELP fusion protein to form the LENN complex. In one example, the LENN complex is prepared with a 5-fold molar excess of prolines plus valines of hydrophobic prosthetic groups of the ELP with respect to cavities of the β-CD at an N:P value from 2 to 5. Also provided is a method of treating bladder cancer in a subject via LENN-mediated delivery of a therapeutic NA cargo, the method comprising administering to the subject a therapeutically effective amount of a composition of LENN complex along with one or more pharmaceutically acceptable carriers, wherein the administering affects intracellular delivery of the condensed NA cargo to bladder tumor cells. In one example, the administering is performed via intravesical infusion.

LENN nanocarriers as described herein are highly tunable and targeted delivery vehicles that can successfully deliver nucleic acid (NA) cargos ranging from ~21 bp siRNA to ~6 kbp pDNA to bladder tumor cell lines. The LENN complexes can efficiently bind to bladder tumor cells within about 30 min of administration and become rapidly internalized to release its NA cargo within about 60 min. LENN nanocarriers are readily adaptable and flexible in their targeting ability, cargo size, nanoparticle formation, and disassembly kinetics. This approach provides an alternative path to lipid nanoparticle formulations that suffer from issues of inefficiency and chemical instability, or viral vectors that are plagued by manufacturing and immune rejection challenges. This agile ELP-based nanocarrier provides an alternative route to nucleic acid delivery using a bio-manufacturable, biodegradable, biocompatible, and highly tunable vehicle capable of targeting cells via engagement with overexpressed cell surface receptors.

### BRIEF DESCRIPTION OF THE DRAWINGS

FIGS. 1A-C presents an illustration of an example LENN complex (including cutaway illustration (FIG. 1B)) and a precursor polyion assembly (FIG. 1C).

FIG. 2 is a flow diagram of intracellular, LENN-mediated delivery of an NA cargo nucleic acid delivery to a transfected tumor cell.

FIG. 3A presents a set of TEM images of siRNA LENN, polyion complex, and constituent formulations using uranyl acetate (UA) as negative stains, where NP2-A-5×N24EGF (col. 1) is a N24EGF:CDPLR₁₀:siRNA LENN complex with a 5-fold molar excess prolines+valines of hydrophobic ELP prosthetic groups with respect to β-CD cavities at N:P=2; NP2-A (col. 2) is a siRNA:CDPLR₁₀ polyion complex with an N:P ratio of 2; N24EGF (col. 3) is a native N24EGF fusion protein without other formulation components; NP4-A-5×N24EGF (col 4) is CDPLR₁₀:5×N24EGF:siRNA LENN complex with a 5-fold molar excess prolines+valines of hydrophobic ELP prosthetic groups with respect to β-CD cavities at N:P=4; and NP4-A (col. 5) is a CDPLR₁₀:siRNA polyion complexes with an N:P ratio of 4.

FIG. 3B presents set of AFM images on cleaved mica surfaces of NP4 polyion complex (row 1) and NP4-A-5×N24EGF LENN complex (row 2).

FIG. 4 presents a reproduced set of TEM images of LENN, polyion complex, and constituent formulations using PTA negative stain, where NP10-A (col. 1) is a pDNA:CDPLR₁₀ polyion complex with an N:P ratio of 10; NP10-A-2×N24EGF (col. 2) is N24EGF:CDPLR₁₀:pDNA LENN complex with a 2-fold molar excess prolines+valines of hydrophobic ELP prosthetic groups with respect to β-CD cavities at N:P=10; and NP10-5×N24EGF (col. 3) is N24EGF:CDPLR₁₀:pDNA LENN complex with a 5-fold molar excess prolines+valines of hydrophobic ELP prosthetic groups with respect to β-CD cavities at N:P=10.

FIG. 5A shows flow cytometry data indicating that binding to T24 human bladder tumor cells requires the presence of EGF on the LENN particle (labeled with Cy5.5) and that LENN particle association with T24 cells increases with incubation time. Different panels show time-based changes in binding and serum competition profiles. The majority of binding occurred within 2 hours. FIGS. 5B and 5C presents box and whisker plots of change in Cy5.5 fluorescence of T24 cell-associated fluorescence 45 min after addition of ELP to CY5.5-dosed NP4 formulations. The concentrations of components were kept similar across different conditions; the polyplexes were prepared then distributed into different wells followed by addition of TNS and stabilization for 45 min; wells with just ELP were prefilled with H₂O followed by TNS addition.

FIGS. 6A and 6B show agarose gel characterization before and after heparin challenges of polyion complexes CD-PLR₁₀:siRNA and CD-PEI_{2.5k}:siRNA at varying N:P ratios and LENN complexes 5×-CDPLR10:N24EGF:siRNA: and 5×-CD-PEI_{2.5k}:N24EGF:siRNA at varying N:P ratios; 100 ng of siRNA loaded in each lane; where, in FIG. 6A, LENN formulations were run at varying N:P ratios, with odd numbered lanes dedicated to 5×-CD-PEI_{2.5k}:5×N24EGF:siRNA with N:P ratios as follows: Lane 1, NP2; Lane 3, NP4; Lane 5, NP6; Lane 6, Lane 7 NP8; and Lane 9, NP10; and even numbered lanes dedicated to CDPLR10:N24EGF:siRNA, with N:P ratios were assigned as follows: Lane 2, NP2; Lane 4, NP4; Lane 6, NP6; Lane 8, NP8; and Lane 10, NP10; and, in FIG. 6B, siRNA polyion complexes at varying N:P ratios, with Lanes 1-5 dedicated to CD-PEI_{2.5k} complexes where N:P ratios were assigned as follows: Lane 1, NP2; Lane 2, NP4; Lane 3, NP6; Lane 4, NP8; Lane 5, NP10; and Lanes 6-10 dedicated to CD-PLR10 complexes where N:P ratios were assigned as follows: Lane 6, NP2; Lane 7, NP4; Lane 8, NP6; Lane 9, NP8; Lane 10, NP10; Lane 11, siRNA alone.

FIGS. 6C-6E show agarose gel characterization before and after heparin challenges of polyion complexes CDPLR₁₀:pDNA and CD-PEI_{2.5k}:pDNA at varying N:P ratios and LENN complexes 5×-CDPLR10:N24EGF:pDNA: and 5×-CD-PEI_{2.5k}:N24EGF:pDNA at varying N:P ratios, where, in FIG. 6C, LENN complexes with 300 ng pDNA with varying N:P ratios, and LENN complex constituents, including Cy5.5-labelled N24EGF (far red emitting), were loaded in lanes as follows: Lane 1, NP8-A-5×N24EGF (targeted); Lane 2, NP8-A-5×N40 (untargeted); Lane 3, pDNA; Lane 4, Cy5.5-N24EGF; Lane 5, NP8-A-Cy5.5-N24EGF (labelled, targeted), where Lanes 4 and 5 were visualized in the far red/near infrared emission channel; in FIG. 6D, characterization of polyion complexes CDPLR₁₀:pDNA and CD-PEI_{2.5k}:pDNA and NP4 5×CD-PLR₁₀:N24EGF LENN complex at N:P=7, where 2×h and 5×h refer to incubation with heparin 2 times and 5 times w/w of pDNA, respectively; in FIG. 6E, stability of pDNA:CD-PEI_{2.5k} polyion complexes and pDNA:CD-PEI_{2.5k}:ELP LENN under heparin challenge (300 ng pDNA) for formulations in Lane 1, pDNA; Lane 2, NP7-B; Lane 3, NP7-B-5×N40 (nontargeting); Lane 4, NP7-B-5×N24EGF (targeted); Lane 5, NP7-B with 0.1×h; Lane 6, NP7-B-5×N40 (untargeted) with 0.1×h; Lane 7, NP7-B-5×N40 (untargeted) with 0.1×h; Lane 8, NP7-B with 2×h; Lane 9, NP7-B-5×N40 (untargeted) with 2×h; Lane 10, NP7-B-5×N40 (untargeted) at NP7 with 2×h.

FIGS. 7A-7H present flow cytometry analyses of T24 and MB49 cell-associated fluorescence after incubation with Cy5.5-ELP LENN (N40, untargeted; N24-EGF, targeted); the ELP content (valine+proline residues) was 5 times the molar ratio of CD-PLR10, where, in FIG. 7A, temporal changes in cell association of LENN (binding+internalization): NP4-5×ELP (Cy5.5-N24-EGF or Cy5.5-N40); in FIG. 7B, addition of serum containing free EGF before addition of NP4-5×N24EGF is shown to have led to a reduction in cell-associated fluorescence compared to treatments with NP4-5×N24-EGF LENN in serum-free media; in FIG. 7C, CD-PLR₁₀ temporal changes in T24 cell-association are reported after incubating with free Cy5.5-N24-EGF and Cy5.5-N40 at the same concentration used in the formulations shown in FIG. 7A; in FIG. 7D, cell-associated fluorescence of NP4-5×ELP LENN is imaged at 1 h and 3 h, where ELP=a physical mixture of Cy5.5-N40 and Cy5.5-N24-EGF during the layer-by-layer deposition step, with ratios ranging from 0:5 to 5:0 such that the total number of moles of (proline+valines) is 5 times that of CD-PLR10 in each case; in FIG. E, changes in cell associated fluorescence are reported upon treatment of MB49 cells with NP2-5×ELP (N24EGF or N40) for 0.5 h, 1 h, and 3 h, where the total number of live cells counted for each condition were kept similar between FIGS. 7D and 7E; in FIG. 6F. changes in MB49 cell-associated fluorescence were reported upon treatment with NP4-5×ELP (N24EGF or N40) for 0.5 h, 1 h, and 3 h; and, in FIG. 6G, confocal analyses of particle internalization is reported for DAPI, pDNA-FITC conjugate, and ELP-Cy5.5 conjugate, and, in FIG. 7H, flow cytometry analyses of cell-associated fluorescence in MB49 after incubation with different formulations of siRNA:CDPLR:Cy5.5-N40, untargeted ELP; N24EGF, targeted ELP at NP4-5×ELP. Different panels show time-based changes in binding and serum competition profiles.

FIGS. 8A and 8B reproduce images from confocal analyses of particle association with MB49 cells after 3 h incubation, where N24EGF and N40 LENN formulations were prepared from FITC-labelled pDNA, CD-PLR10, and Cy5.5-labelled ELP; nuclei were stained with DAPI. LENN formulated with untargeted Cy5.5-N40 displayed very weak cellular fluorescence for either the Cy5.5-ELP or FITC-pDNA associated fluorescence channels; whereas LENN formulated with N24EGF showed extensive cellular association, similar to that of Lipofectamine. Any applied image correction had been normalized across all the conditions.

FIG. 9 shows relative expression levels of HIF-1α at 70 h post transfection as determined by RT-PCR. YC-1 is a small molecule inhibitor of HIF-1α. Formulations with ELP were made using a 5×ELP:CD ratio. All the conditions were normalized against normoxia and scaled by their exerted toxicity.

FIGS. 10A-10C reproduce images from confocal analysis of GFP expression 48 h post-transfection of MB49 cells (10A and 10B) and RAW264.7 cells (10C) with various pDNA:CDPLR₁₀ formulations. Lipofectamine was used as a positive control for transfection and any applied image correction had been normalized across all the conditions.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of involvement or the severity of the disease or disorder, the response of the individual patient, the particular compound administered, the mode of administration, the bioavailability characteristics of the preparation administered, the dose regimen selected, the use of concomitant medication, and other relevant circumstances.

As used herein, the term “patient” includes human and non-human animals such as companion animals (dogs and cats and the like) and livestock animals. Livestock animals are animals raised for food production. The patient to be treated is preferably a mammal, in particular a human being.

As used herein, “nucleic acids” (NA) refers to a polymeric form of nucleotides of any length and nucleic acids may be referred to interchangeably herein as “nucleic acids,” “NA,” “oligonucleotides,” “nucleic acid molecules,” “nucleic acid materials,” “nucleic acid sequences,” “biopolymers,” or “polynucleotides.” Nucleic acids consistent with the present disclosure may comprise DNA, RNA, DNA: RNA hybrids, recombinant DNA, or hybrid DNA, and may be single-stranded, double-stranded, or multiple stranded. One strand of a nucleic acid also refers to its complement. Example nucleic acids herein may also include polynucleotide analogues, amplicons, conjugates, and substitutions, polynucleotides, crosslinked polynucleotides, polynucleotide complexes, and non-natural nucleic acids, including, but not limited to, dideoxynucleotides, nucleosides, morpholinos, peptide nucleic acids, or chemically-modified polynucleotides, including ribose-modified, biotinylated, methylated, aminated, deaminated, alkylated, benzylated, or fluorophore-labeled polynucleotides.

Example DNA includes nuclear DNA (nDNA), mitochondrial DNA (mtDNA), DNA-based vectors, such as plasmid DNA (pDNA), minicircle DNA (mcDNA), and linear DNA amplicons, antisense oligonucleotides (ASO), unmethylated cytosine-phosphate-guanine (CpG) oligodeoxynucleotides, DNA enzymes, and DNA methyltransferase inhibitors. Example RNA includes messenger RNA (mRNA), small-interfering RNA (siRNA), short hairpin RNA (shRNA), microRNA (miRNA), piwi-interacting RNAs (piRNA), enhancer RNA (eRNA), long non-coding RNA (lncRNA), long intergenic non-coding RNA (lincRNA), small activating RNA (saRNA) small nucleolar RNA (snoRNA), circular RNA (circRNA), ribozymes, short open reading frame (sORF)-containing RNA, and CRSPR-Cas9-related RNA, e.g., crspr RNA (crRNA), guide RNA (gRNA), single guide RNA (sgRNA), and tracr RNA.

In one aspect, a method of preparing an elastin-like polypeptide (ELP) carrier system for delivery of a nucleic acid cargo to overexpressing EGFR (epidermal growth factor receptor) tumor cells is provided, including the steps of: complexing nucleic acid material of the nucleic acid cargo with decaarginine-β-cyclodextrin (CD) to yield a CD-BP polyplex cargo; and coating the CD-BP polyplex cargo, at least in part, with an ELP-epidermal growth factor (ELP-EGF) fusion protein to yield the ELP carrier system. ELP can be a (VPGXG) n construct, wherein n represents the number of pentapeptide repeats and X represents a predetermined amino acid. For instance, X can be valine and n=24. In examples, the ELP carrier system can be prepared with a nucleic acid cargo one or more of: siRNA, pDNA, miRNA, mRNA, and dsDNA. In the same or other examples, the nucleic acid cargo is effective in treating overexpressing EGFR bladder tumor cells.

In another aspect, an ELP carrier system is provided that includes (1) an NA material complexed with decaarginine-β-cyclodextrin (CD); and (2) an outer coating wrapping, at least in part, the CD-condensed nucleic acid material with one or more ELP-epidermal growth factor (ELP-EGF) fusion proteins. The ELP can be (VPGXG) n construct, wherein n represents the number of pentapeptide repeats and X represents a predetermined amino acid. For instance, X can be valine and n=24. In examples, the ELP carrier system condenses a nucleic acid cargo one or more of siRNA, pDNA, miRNA, mRNA, and dsDNA. In examples, the ELP carrier system including pharmaceutical compositions of same, is formulated for target delivery of a therapeutic NA cargo to overexpressing EGFR bladder tumor cells

In certain embodiments, the ELP carrier system can be a layer-by-layer ELP-nucleic acid (NA) nanoparticle (LENN) complex, according to which the LENN complex can include a polyion complex core wrapped, at least in part, by an ELP fusion protein coating. In examples, the polyion complex core includes a nucleic acid cargo condensed by an oligocation and the ELP fusion protein comprises an EFP linked directly or indirectly to a co-expressed targeting ligand. In examples the same or other examples, the nucleic acid material can comprise one or more of: siRNA, pDNA, miRNA, mRNA, and dsDNA. In the same or still other examples, the oligocation is decaarginine-β-cyclodextrin (CDPLR10), 2.5 kD polyethyleneimine-modified β-cyclodextrin (CDPEI_{2.5k}), or other oligocationic cyclodextrin derivatives.

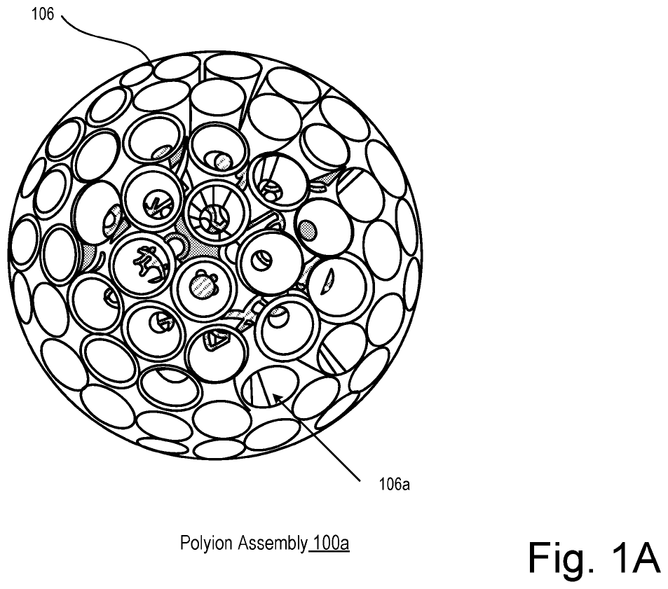

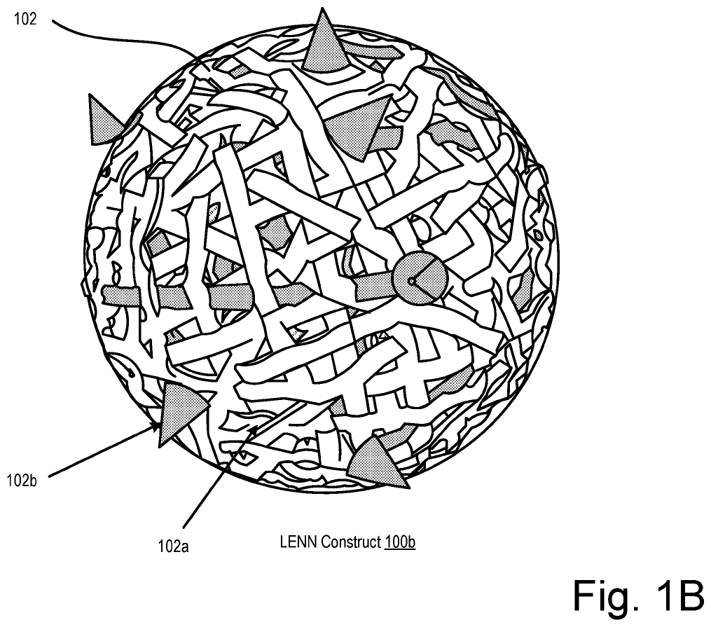

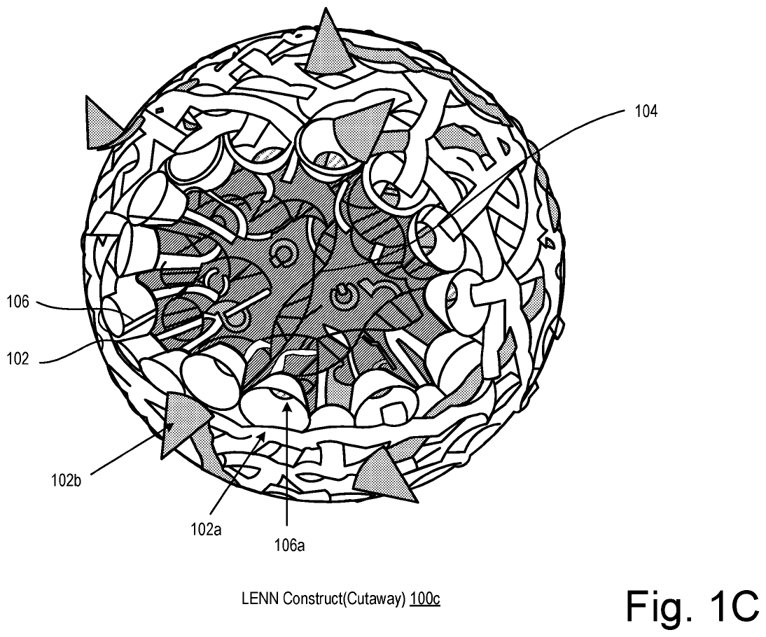

FIGS. 1A-1C provides an illustration of an example LENN complex 100b formed using a layer-by-layer (LbL) deposition technique, as described herein, along with a polyion core 100a intermediate. The LENN complex 100b of FIG. 1B is formed, first, by condensing an NA cargo with a β-cyclodextrin (CD) oligocation, e.g., decaarginine-modified β-cyclodextrin (CDPLR10) or 2.5 kD polyethyleneimine-modified β-cyclodextrin (CDPEI_{2.5k}), to form polyion core 100a (FIG. 1B) in which cavities 106a supported by the molecular structure of the β-cyclodextrin are self-organized into a host surface 106b enveloping within it the condensed NA cargo 104 (shown in the cutaway (FIG. 1C)) and having size dependent stoichiometry that facilitates host:guess interaction between nonpolar prosthetic groups of the ELP and the hydrophobic cavity interiors. The polyion core 100a is then wrapped with an ELP fusion protein coating 102 of ELP 102a linked directly or indirectly to a co-expressed targeting ligand 102b, e.g., epidermal growth factor (EGF), to form an electrostatically condensed LENN complex 100b capable of crossing the anionic cell membrane for intracellular delivery of the condensed NA cargo 104.

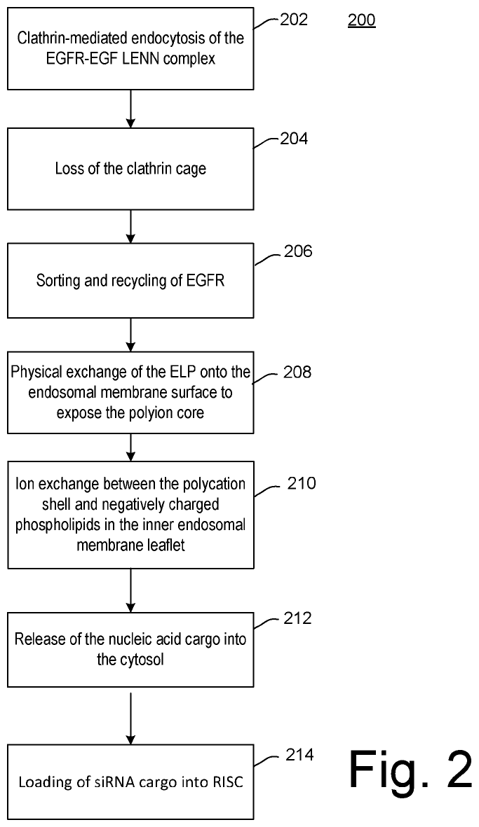

With reference to the flow diagram of FIG. 2, LENN-mediated intracellular delivery 200 of an NA cargo in an example ELP-EGF: CD: NA LENN complex may involve, in 202, clathrin-mediated endocytosis of the ELP-EGF: CD: NA LENN complex, in 204, loss of the clathrin cage, in step 206, sorting and recycling of EGFR, in 208, physical exchange of the ELP onto the endosomal membrane surface to expose the polyion core, in 210 and 212, ion exchange between the polycation core and negatively charged phospholipids in the inner endosomal membrane leaflet to release the nucleic acid cargo into the cytosol, and, in 214, loading of siRNA cargo into RISC.

This highly adaptable layer-by-layer (LbL) deposition technique provides facile modulation at various points in the formulation process to enable tunability of LENN carrier systems properties for optimal cell binding, internalization, and intracellular cargo release across a wide range of NA cargo sizes. A core design principle of the present LENN system is the programmable nanoparticle stability afforded by the cooperativity of multiple host-guest interactions between the ELP prosthetic groups and the CD cavities to provide a tunable balance between the stability need to reach the biological target site and the capacity for programmed disassembly within the target cell to enable efficacious bioactivity (see FIG. 2). Moreover, the process of wrapping a CD oligocation-condensed NA cargo with ELP serves a number of important functions in the context of bladder cancer therapeutics. For instance, the high cooperativity of the multiple host:guest interactions between the nonpolar prosthetic groups of the ELP and the hydrophobic β-cyclodextrin cavities promote further condensation of the NA cargo into stable LENN-based electrostatically condensed polyplexes. The ELP wrapping also acts to shield excess positive charge on the electrostatically condensed polyplexes to limit non-specific adsorption of the LENN-based polyplexes to the glycans-rich bladder lumen. ELP may be co-expressed as fusion proteins with a variety of targeting ligands to enhance tumor selectivity.

Table 1 summarizes some of the key distinctions between the present ELP-based LbL approach and the prior art LNP approach and viral vectors approach. Two main drivers for pursuing an ELP-based approach are the biocompatibility and non-immunogenicity of ELP compared to viral and the obviation of the chemical instability found in lipid formulations using ionizable lipids.

TABLE 1
Comparison among LENN-based, LNP, and viral NA delivery systems

| Vector Type | | LENN | LNP | Virus |
|---|---|---|---|---|
| Properties | Condensation | Cyclodextrin- | Protonated ionizable | Nucleocapsid |
| | element | polycation | lipid self-assembly | proteins |
| | Stealth element | ELP | PEG (polydisperse | — |
| | | (monodisperse | synthetic polymer) | |
| | | polypeptide) | | |
| | Targeting element | Receptor-specific | Receptor-specific | Receptor- |
| | | ligand | ligands (in some | binding |
| | | | cases) | domains of |
| | | | | capsid proteins |
| | Release element | Polycationic | Endosomal membrane | Fusogenic |
| | | oligopeptide and | permeabilization | peptides |
| | | β-CD | mediated by ionizable | |
| | | | lipids | |
| | Cargo size | siRNA-pDNA | siRNA-pDNA | Usually, ≤4 kb |
| | Liabilities | Protein stability | Chemical instability | Immunogenic |

In the context of bladder cancer, the targeting ligand can be any peptide or protein capable of selective interaction with a targeted membrane protein (or glycoprotein) implicated in cancer behavior the expression of which may be characterized as either (1) overexpressed (or upregulated) in bladder tumors compared to normal tissues or (2) aberrantly expressed in bladder tumors but not in normal tissues. By way of example, the targeting ligand can be a peptide or protein capable of selective interaction with an upregulated integrin, receptor tyrosine kinase (RTK), or G-protein coupled receptor. For instance, the targeting ligand can be a peptide or protein capable of selective interaction with one of αEβ7 integrin, integrin α2β1, integrin β8, EGFR, FGFR, HER 2, VEGFR-2, or chemokine receptor 7 (CCR7). In one example, in which the membrane protein is EGFR, the ELP fusion protein can be N24EGF. By way of further example, the targeting ligand can be a peptide or protein capable of selective interaction with an aberrantly expressed cell surface adhesion molecules (CAMs). For instance, the targeting ligand can be a peptide or protein capable of selective interaction with either NECTIN-4 or the surface adhesion glycoprotein TROP-2.

LENN complexes herein support a wide range of NA sizes—from as small as 20 bp non-coding RNAs to as large as 10 kpb pDNAs. In examples, condensed NA cargos may include NA between about 20-40 bp in length (e.g., miRNAs, siRNAs), about 40-100 bp in length (e.g., shRNAs), about 100 to 500 bp in length (e.g., mtRNA, nDNA protein coding sequences), about 300 bp to about 4 kbp in length (mRNAs), or about 5 kbp or greater in length (e.g., pDNAs).

In certain illustrative embodiments, a LENN complex may include a therapeutic NA cargo. By way of example, therapeutic RNAs may include siRNAs, shRNAs, miRNAs, saRNAs, crRNAs, ribozymes, antagonist RNA aptamers, and cell type-specific RNA aptamers. Example therapeutic DNAs include pDNA, mcDNA, and other DNA-based vectors, ASOs, CpG oligodeoxynucleotides, DNA methyltransferase (DNMT) inhibitors.

LENN complex formulated in a composition for intracellular delivery of a condensed therapeutic NA cargo to a bladder tumor. The composition may include one or more pharmaceutically-active carriers.

Therapeutic NAs is any nucleotide material that when introduced into tumor cells produces a therapeutic benefit. Therapeutic benefits produced based on various molecular methods, including, e.g., silencing or downregulating overexpressed or aberrantly expressed genes implicated in tumor promotion; activation or upregulation of expression of tumor suppressor genes; demethylation of hypermethylated tumor suppressor genes; induced degradation or translation repression of mRNA transcripts of genes implicated in tumor promotion; or inhibition of protein: protein or receptor-ligand interactions involved in signal transduction along dysregulated oncogenic pathways.

The ELP consistent for use with a LENN Carrier system of the disclosure may be a (VPGXG) n construct, where n represents the number of pentapeptide repeats and X represents a predetermined amino acid. By way of example, n may be 5≤n≤50, 10≤n≤40, 10≤n≤30, 15≤n≤25, n=20, n=21, n=22, n=23, n=24, n=25, n=26, n=27, n=28, n=29. In examples, X may be any amino acid other than proline. In one example construct, X is valine, and n=24.

The EGF may be any protein that stimulates cell growth and cell differentiation. In one embodiment, the EGF is a protein murine epidermal growth factor.

Oligocations for use consistent with the technology may be a β-cyclodextrin (CD) oligocation, e.g., decaarginine-modified β-cyclodextrin (CD-PLR10) or 2.5 kD polyethyleneimine-modified β-cyclodextrin (CD-PEI_{2.5k}). Oligocations may also include histones, polyamines, spermidines, spermines, or inorganic cobalt hexamine.

The targeting ligand may be a peptide or protein capable of selective interaction with a targeted membrane protein (or glycoprotein) the expression of which may be characterized as either (1) overexpressed (or upregulated) in bladder tumors compared to normal tissues or (2) aberrantly expressed in bladder tumors but not in normal tissues. Common types of upregulated membrane protein receptors implicated in cancer behavior include integrins, receptor tyrosine kinases (RTKs), G-protein coupled receptors (GPCRs). In that regard, targeting ligands appropriate for use with the LENN technology herein may be any ligand capable of selective interaction with an upregulated integrin, RTK, or GPCR. For example, targeting ligands for implementation in LENN complexes for uses consistent with the disclosure may include ligands capable of selective interaction with one of αEβ7 integrin, integrin α2β1, or integrin β8 membrane glycoproteins upregulated in bladder tumors. Other targeting ligands for implementation in LENN complexes for uses consistent with the disclosure may include ligands capable of selective interaction with one of EGFR, FGFR, HER 2, or VEGFR-2 RTK family proteins. Other targeting ligands for implementation in LENN complexes for uses consistent with the disclosure may include ligands capable of selective interaction with upregulated PGCRs associated with bladder cancer including chemokine receptor 7 (CCR7) PGCR family protein. Still other targeting ligands for implementation in LENN complexes for uses consistent with the disclosure may include ligands capable of selective interaction with upregulated CAMs associated with bladder cancer, including Nectin-4 and the cell surface adhesion glycoprotein, TROP-2.

Aberrantly expressed membrane proteins include proteins expressed from germline-specific genes activated in bladder tumors but not in normal tissues. These include, e.g., cell surface adhesion molecules (CAMs) such as NECTIN-4 and the surface adhesion glycoprotein TROP-2.

In yet other illustrated embodiments, methods for treating a patient with bladder cancer may include the step of administering to the patient a therapeutically effective amount of a composition of a LENN complex, together with one or more pharmaceutically acceptable carriers. Administering may be performed according to any of a number of accepted techniques useful in promoting localized, minimally-systemic intracellular delivery of the LENN complex to a bladder tumor. By way of example, administering may be performed via intravesical infusion. Administering may also be performed through intravenous, intramuscular, subcutaneous, inhalation, or intranasal routes.

### EXAMPLES

The following examples serve to illustrate the present disclosure. The examples are not intended to limit the scope of the claimed invention in any way.

### Polyion Complexation and Layer-by-Layer ELP:Nucleic Acid Nanoparticle (LENN) Formulation

The following stocks were prepared and used for all formulations unless stated otherwise: nucleic acid cargo (siRNA or pDNA) in RNAase-/LPS-free water (0.1 mg/mL) and polycation (CD-PLR10 (A) or CD-PEI_{2.5k} (B)) in 18 MΩ H₂O such that the concentration of positive charges (secondary amines in PEI_{2.5k} and guanidinium groups in R₁₀) was 10 mM. First, nucleic acid and oligocations were vortex-mixed for 30 sec at the desired N:P ratio and incubated at 20° C. for 30 min. N24EGF (targeted) or N40 (untargeted) ELP was added to the polyion complexes with vortex mixing to form LENN. Varying amounts of ELP were added to produce defined molar ratios of the valine and proline residues in the LENN ELP layer. Formulation compositions herein are represented by suffixing the molar ratio and identity of the ELP, such that NP4-A-5×N24EGF refers to a nanoparticle formulation where the amount of ELP added has a 5-fold molar excess of valine+proline residues relative to CDPLR₁₀. Tables 2A and B provide a list of prepared LENN complexes and expansion on nomenclature. HIF-1α siRNA (NM 010431.2) (ThermoFisher) was used for the siRNA cargo. GFP-NLS plasmid was used for the pDNA cargo.

TABLE 2A
Prepared siRNA · LENN Complexes.
siRNA · LENN Complexes

| | |
|---|---|
| NP2-A-5x N24EGF | N24EGF:CDPLR₁₀:siRNA LENN |
| | complex with 5-fold molar excess prolines + |
| | valine of hydrophobic ELP prosthetic groups |
| | with respect to β-CD cavities at N:P = 2 |
| NP2-B-5x N24EGF | N24EGF: CDPEI_{2.5k}:siRNA LENN |
| | complex with 5-fold molar excess prolines + |
| | valine of hydrophobic ELP prosthetic groups |
| | with respect to β-CD cavities at N:P = 2 |
| NP4-A-5x N24EGF | N24EGF:CDPLR₁₀:siRNA LENN |
| | complex with 5-fold molar excess prolines + |
| | valine of hydrophobic ELP prosthetic groups |
| | with respect to β-CD cavities at N:P = 4 |
| NP4-B-5x N24EGF | N24EGF: CDPEI_{2.5k}:siRNA LENN |
| | complex with 5-fold molar excess prolines + |
| | valine of hydrophobic ELP prosthetic groups |
| | with respect to β-CD cavities at N:P = 4 |
| NP6-A-5x N24EGF | N24EGF:CDPLR₁₀:siRNA LENN |
| | complex with 5-fold molar excess prolines + |
| | valine of hydrophobic ELP prosthetic groups |
| | with respect to β-CD cavities at N:P = 6 |
| NP6-B-5x N24EGF | N24EGF: CDPEI_{2.5k} siRNA LENN |
| | complex with 5-fold molar excess prolines + |
| | valine of hydrophobic ELP prosthetic groups |
| | with respect to β-CD cavities at N:P = 6 |
| NP8-A-5x N24EGF | N24EGF:CDPLR₁₀:siRNA LENN |
| | complex with 5-fold molar excess prolines + |
| | valine of hydrophobic ELP prosthetic groups |
| | with respect to β-CD cavities at N:P = 8 |
| NP8-B-5x N24EGF | N24EGF: CDPEI_{2.5k} siRNA LENN |
| | complex with 5-fold molar excess prolines + |
| | valine of hydrophobic ELP prosthetic groups |
| | with respect to β-CD cavities at N:P = 8 |
| NP10-A-5x N24EGF | N24EGF:CDPLR₁₀:siRNA LENN |
| | complex with 5-fold molar excess prolines + |
| | valine of hydrophobic ELP prosthetic groups |
| | with respect to β-CD cavities at N:P = 10 |
| NP10-B-5x N24EGF | N24EGF:CDPEI_{2.5k} siRNA LENN |
| | complex with 5-fold molar excess prolines + |
| | valine of hydrophobic ELP prosthetic groups |
| | with respect to β-CD cavities at N:P = 10 |

TABLE 2B
Prepared pDNA · LENN Complexes
pDNA · LENN Complexes

| | |
|---|---|
| NP7-B-5x N24EGF | N24EGF: CDPEI_{2.5k}:pDNA LENN complex |
| | with a 5-fold molar excess prolines + valines |
| | of hydrophobic ELP prosthetic groups with |
| | respect to β-CD cavities at N:P = 7 |
| NP8-A-5x N24EGF | N24EGF:CDPLR₁₀:pDNA LENN complex |
| | with a 5-fold molar excess prolines + valines |
| | of hydrophobic ELP prosthetic groups with |
| | respect to β-CD cavities at N:P = 8 |
| NP10-A-2x N24EGF | N24EGF: CDPLR₁₀:pDNA LENN complex |
| | with a 2-fold molar excess prolines + valines |
| | of hydrophobic ELP prosthetic groups with |
| | respect to β-CD cavities at N:P = 10 |
| NP10-A-5x N24EGF | N24EGF:CDPLR₁₀:pDNA LENN complex |
| | with a 5-fold molar excess prolines + valines |
| | of hydrophobic ELP prosthetic groups with |
| | respect to β-CD cavities at N:P = 10 |

### Agarose Gel Electrophoresis

Agarose gel electrophoresis was used to evaluate the nucleic acid complexation status in various formulations. In brief, agarose gels (2% for siRNA; 1% for pDNA) were cast by microwaving appropriate amount of agarose in pH 7.4 Lithium Acetate Borate buffer (LAB buffer) until the solution turned clear and then cooled to touch before addition of GelRed at a 1:10,000 dilution. The warm gel+dye solution was carefully poured to avoid any bubbles in the sample lanes in a gel holder with a comb of wells. Samples were diluted with a 6× sample loading buffer (without SDS). Nucleic acid complexation efficiency was determined by comparing the fluorescence signatures of GelRed-accessible nucleic acid binding relative to free nucleic acid as a positive control using the same nucleic acid concentration in each well. The gel was run at 200 V for 20 min and then imaged with a Bio-Rad Chemidoc Touch Imaging System.

### Heparin Challenge

Heparin was used to test the stability of LENN formulations by agarose gel electrophoresis. The polyion complexes and LENN formulations were subjected to different ratios of heparin relative to the nucleic acid cargo (w/w) for 30 min. Two different heparin stocks were made to probe stability at both low and high molar equivalence heparin using equal volumes of the appropriate stock to mitigate dilution effects on complexation/decomplexation behavior.

### Atomic Force Microscopy

A Veeco MultiMode Atomic Force Microscope with Nanoscope V Controller was used to obtain AFM images in tapping mode. AFM tips were 75 kHz with 2.8 N/m force constant. Aqueous solutions of the nucleic acid formulations (0.5 μg/20 μL) were drop-cast onto mica and allowed to evaporate overnight before imaging.

### Transmission Electron Microscopy

Nucleic acid formulations were diluted 3-4-fold with 18 MΩ H₂O before casting 3 μL of the solution onto the TEM grid. After 3-5 sec, the grid was blotted and then 3 μL of 1% uranyl acetate or phosphotungstic acid was added and incubated for 10-15 sec before blotting away the excess and drying overnight in a dessicator before imaging. Images were acquired using a FEI Tecnai G2 20 electron microscope, equipped with a LaB₆ source and a Gatan US1000 2K CCD, operating at 100-200 kV.

### Dynamic Light Scattering

Particle size, size distributions, and zeta potential of the different formulations were determined using a Malvern Zetasizer Nano ZS instrument. After formulation of 2 μg nucleic acids into polyion complexes or LENN, 700 μL of 10 mM HEPES+10 mM NaCl solution (pH=7) was added for size and zeta measurements. All measurements were made in triplicate.

### Cell Culture

Mycoplasma-free T24 human (procured from ATCC) and MB49 mouse (gift from Prof. Timothy Ratliff, Purdue Institute for Cancer Research) urothelial carcinoma cell lines were used to evaluate the performance of various NA complexes. T24 cells were cultured in McCoy's medium supplemented with 10% FBS at 37° C. and 5% CO₂. MB49 cells were cultured in DMEM medium supplemented with 10% FBS, 1% penicillin/streptomycin under the same conditions as the T24 cells.

### Transfection to Bladder Tumor Cell Lines and Measurement of Toxicity

Cells were seeded at a density of 50,000 cells/well in 12-well plates overnight in serum-containing media and allowed to attain 30-50% confluency. Serum-containing media was replaced with serum-free media 2-3 h before the addition of the NA formulations and incubation at 37° C. and 5% CO₂ for 5 h. The NA treatments were then removed, the cells supplemented with serum containing complete media, and incubated for varying times before measuring toxicity using the CellTiter 96® AQueous assay as per the recommended protocol. Typical cell treatment amounts were 2 μg for pDNA and 0.54 μg for siRNA.

### Cell Binding Assessment by Flow Cytometry

The nanoparticles were formulated with Cy5.5-conjugated ELP and the cells prepared for transfection as described above. For serum competition studies, FBS was added to the cells to obtain a 20% (v/v) solution before adding the NA formulations. After incubating the NA complexes with cells for different time periods at 37° C. under 5% CO₂, cells were washed with 1×PBS, followed by resuspension in 1 mL flow cytometry staining buffer (1% BSA and 0.1% NaN₃) and filtered through tubes with a cell strainer cap to remove multi-cellular clusters. Cell slurries from various treatments were evaluated using a BD LSRFortessa™ cell analyzer using the APC-Alexa 700 channel (λₑₓ: 640 nm/λₑₘ: 710 nm) and the data processed using FCS Express 7.

### Cell Binding and Internalization Monitored by Confocal Microscopy

Poly-L-lysine coated slides (6-well plates) were seeded with 150,000 cells/well and incubated in serum-containing media until ~50% confluency was reached. Time-based internalization studies were performed for the same conditions and concentrations as the binding studies. Post-treatment, cells were washed with 1×PBS and then incubated in 2% paraformaldehyde for 10-15 min (5 min when analyzing GFP expression), followed by 2 more washes with 1×PBS. The coverslips were then mounted on glass slides using Ibidi® mounting media (with or without DAPI) and analyzed with a Nikon AIR-MP microscope using the fluorescence channels (DAPI, TRITC, FITC, Cy5.5) relevant for the particular experiment.

Fluorescence Studies Using Fluorimetric 2-p-toluidinylnaphthalene-6-sulfonate (TNS)

siRNA and CD-PLR10 polyplexes were prepared, distributed into 384-wells under various conditions, 20 μM TNS added, and the solutions mixed by pipetting to give a TNS concentration of 10 μM (final concentrations of CD-PLR₁₀≈0.1 mM and siRNA≈0.05 μg/μL). After 45 min at 20° C., the fluorescence intensity was monitored [ex: 320 nm; em: 500 nm] before addition of N24-EGF to give a range of concentrations (1×-0.70 μM, 2×-1.41 μM, 3×-2.05 M, 4×-2.66 μM, 5×-3.23 M) and pipette-mixed before monitoring the fluorescence intensity again.

### Example 1: Biophysical Characterization of Condensed siRNA Cargos in Polyion-Complexes and LENN

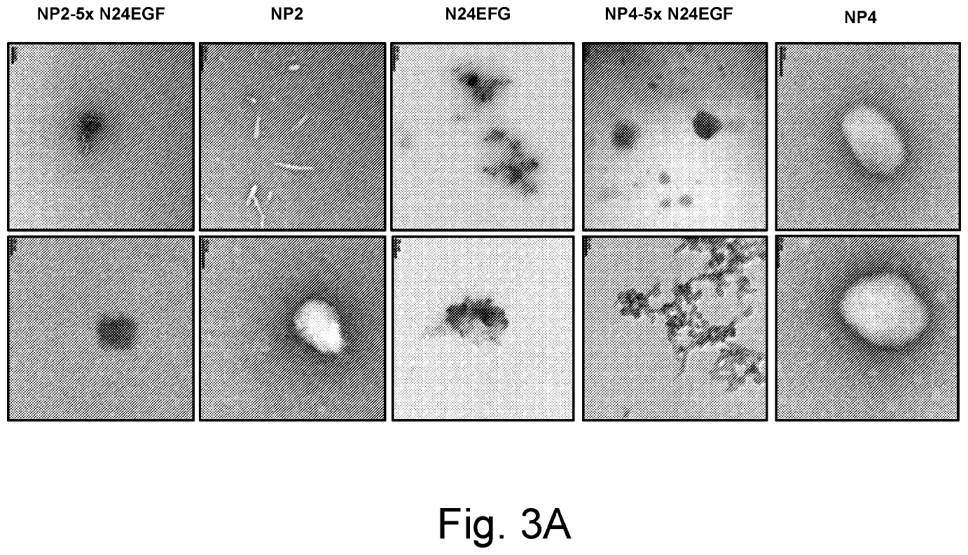

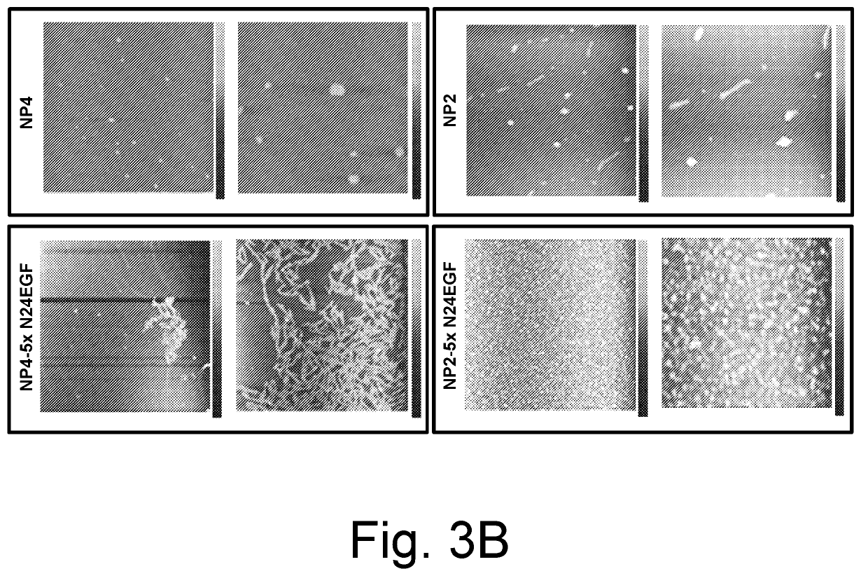

With reference to FIG. 3A, transmission electron microscopy (TEM) images were collected after staining sample LENN complexes with 1% uranyl acetate. The initial CDPLR10:siRNA polyion complexes appear as a mixture of spheres and ellipsoids with diameters of ~50 nm. As shown in TEM images of FIG. 3A, after addition of N24EGF to the polyion complexes, the LENN complexes displayed a mixture of 200-300 nm aggregates that appeared as 2-D networks of smaller assemblies and spherical 50 nm particles.

Since globular and network structures were also observed for N24EGF alone (FIG. 3A, column 3), the ‘di-block’ amphipathic N24EGF sequence was observed to have similar aggregation behavior other di-block ELP designs. With reference to FIG. 3B, the sizes and size distributions of nanoparticles observed by TEM were also corroborated by Atomic Force Microscopy (AFM), which shows ELP condensing around the nucleic acid sample and network-like structures with other condensates. These observations suggest that ELP can self-associate, thus highlighting the importance of striking the right stoichiometric balance of ELP: β-cyclodextrin to promote host:guest interaction between nonpolar ELP prosthetic groups and hydrophobic cavities of β-cyclodextrin over self-association.

### Example 2: Biophysical Characterization of Condensed pDNA Cargos in Polyion-Complexes and LENN

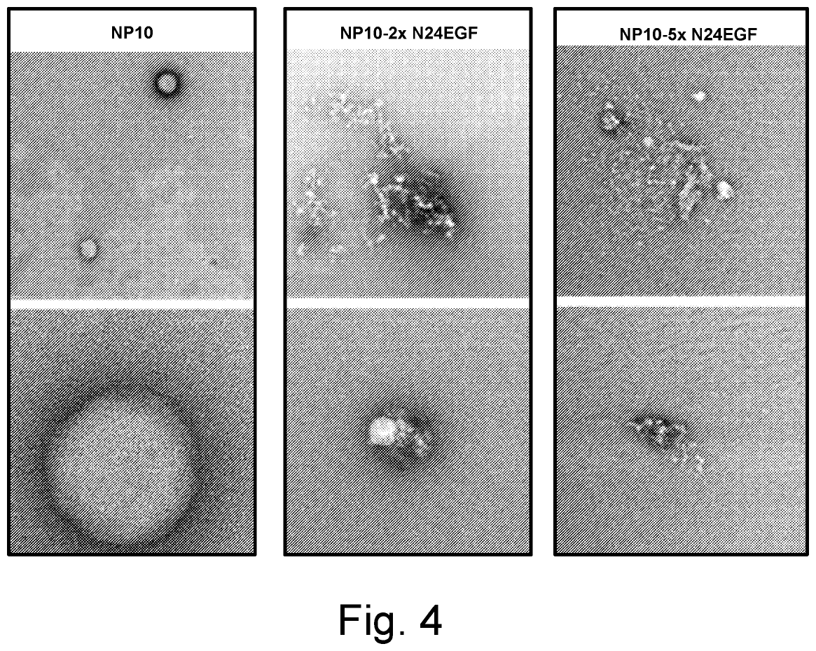

With reference to FIG. 4, sample pDNA LENN complexes (NP10) (column 1) were observed to be ~80 nm and more uniformly spherical than the siRNA complexes at appreciably higher N:P ratios than reported for other pDNA delivery systems. The addition of N24EGF (columns 2 and 3) did not lead to any further observable compaction, however, the particle size distribution became broader, ranging from 30-200 nm, regardless of the N:P ratio or polyion complex:ELP ratio employed.

### Example 3: Cy5.5 Fluorescence Analysis of LENN Assembly Behavior

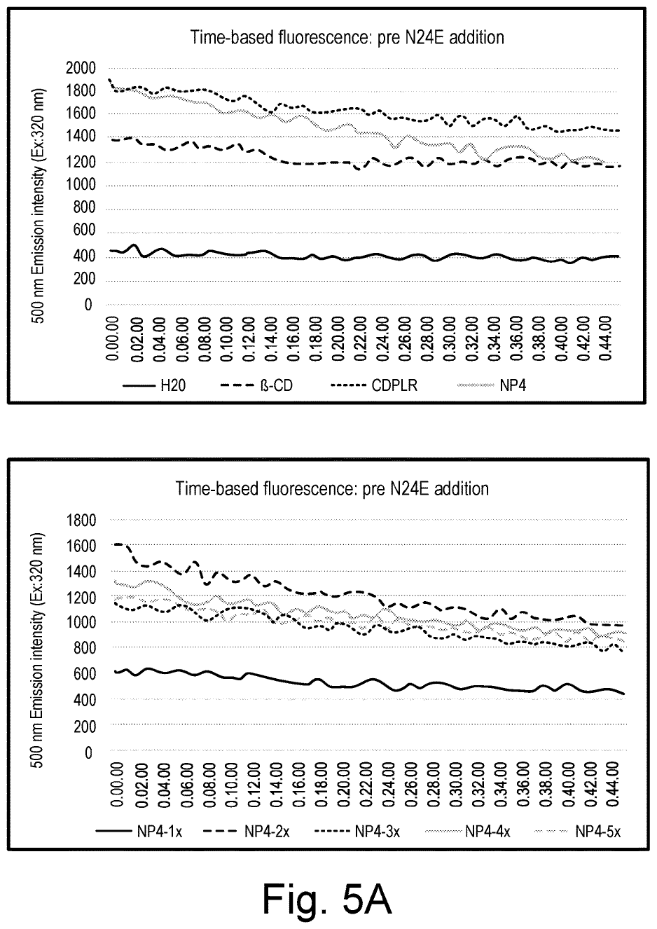

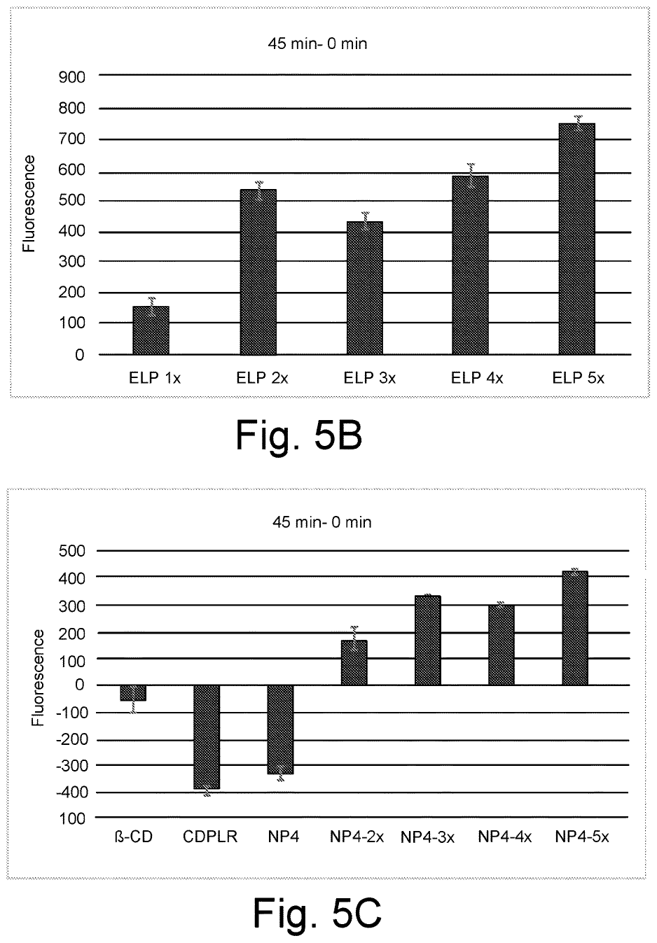

A Cy5.5 fluorescence-based assay was performed to report on assembly behavior during LENN complex formation. With reference to FIG. 5A, when Cy5.5 was added to unmodified β-CD, CDPLR10, and NP4 polyion complex, the fluorescence intensity increased compared to the aqueous controls at the same probe concentration, with the highest observed increases observed for CDPLR10 and NP4. The increase in fluorescence intensity was likely due to the combined effect of Cy5.5 host:guest inclusion within the hydrophobic β-CD cavity and enhanced binding due to ion pairing of the TNS sulfonate with the CDPLR₁₀ polycationic ponytail. As shown in FIGS. 5B and 5C, after addition of N24EGF:ELP, the change in fluorescence between t=0 min and t=45 min of incubation was positively correlated with the amount of ELP added. This change of fluorescence was attributed to an increased hydrophobic environment in the vicinity of the probe due to cooperative binding of the ELP prosthetic groups to preformed polyion complexes.

### Example 4: Agarose Gel Analysis of Stability of CD-Condensed siRNA and pDNA Polyion Complexes

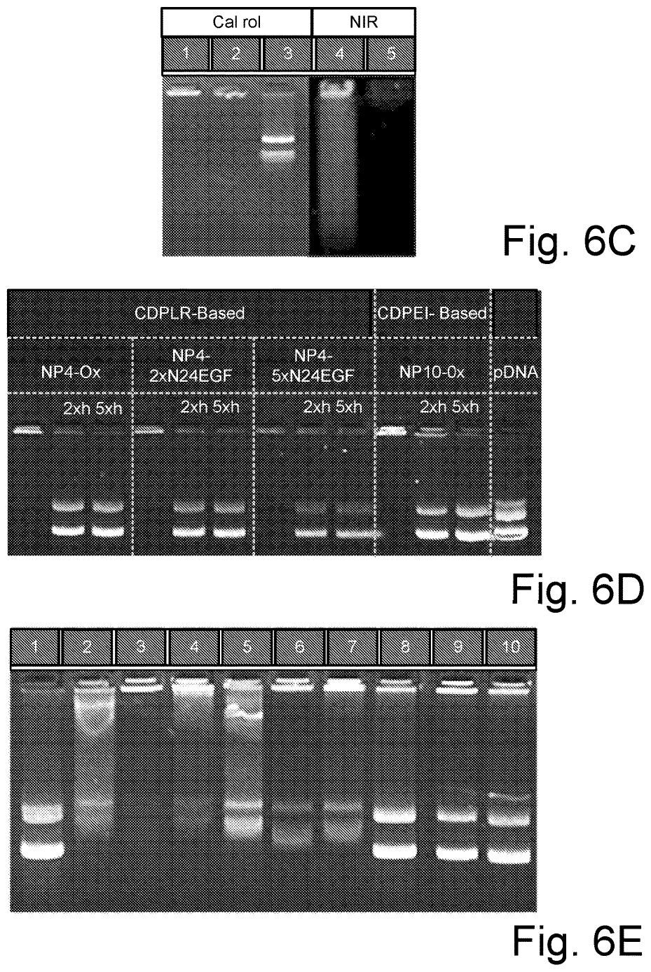

With reference to FIGS. 6A-6E, agarose gel characterization of siRNA-LENN formulations (FIG. 6A) and pDNA·LENN formulations (FIG. 6B) demonstrated that both siRNA and pDNA formed stable complexes with CDPLR₁₀ and CDPEI_{2.5k}, where CDPLR₁₀ (FIGS. 6A and 6B, even numbered lanes) were found to form more stable polyion complexes than CDPEI_{2.5k} (FIGS. 6A and 6B, odd numbered lanes). The stability of the polyion complexes and LENN toward heparin challenge was also tested as a function of encapsulated NA:heparin ratio. Agarose gel characterization as provided in FIGS. 6D-6E showed that layer-by-layer coating of the polyion complex with ELP imparts an enhanced resistance toward LENN complex disassembly in the presence of heparin. From these findings, it was concluded that host:guest interactions between the CD-PLR10 cavities and the ELP prosthetic groups confer a charge shielding effect on the polyion complexes, consistent with observed reduction in zeta potential for these complexes (See Table 3, NP4 vs. NP4-5×N24EGF).

TABLE 3
Observed diameters and zeta
potentials of polyion complexes and LENN

| Formulation | Dₐᵥₑ (nm) | zetaₐᵥₑ (mV) |
|---|---|---|
| NP2 | 159 | −0.04 |
| NP2-5x N24EGF | 136 | −0.04 |
| NP4 | 116 | 9.8 |
| NP4-5x N24EGF | 119 | 1.7 |

Because the heparin environment mimics the bladder lumen environment and its dense coverage with negatively charged glycans, the heparin challenge is commonly accepted and highly reliable stability test for bladder cancer therapies. As such, a strong inference could be made that the observed charge shielding effect would confer similar stability to LENN-based therapeutics administered in vivo.

Differential migration behavior for the free and complex-bound forms of Cy5.5-N24EGF in an electric field further demonstrated the arrangement of ELF wrapping the polyion complex (FIG. 6C, Lane 4). The net negative charge within the EGF domain of N24EGF promotes migration toward the cathode under an applied potential, whereas this movement is abrogated when pDNA:CDPEI2.5k polyion complexes are wrapped with N24EGF via LbL deposition.

### Example 5: Quantitative Flow Cytometry Analysis of Transfection of T24 (Human) and MB49 (Murine) Bladder Cancer Cells with siRNA-LENN Complexes

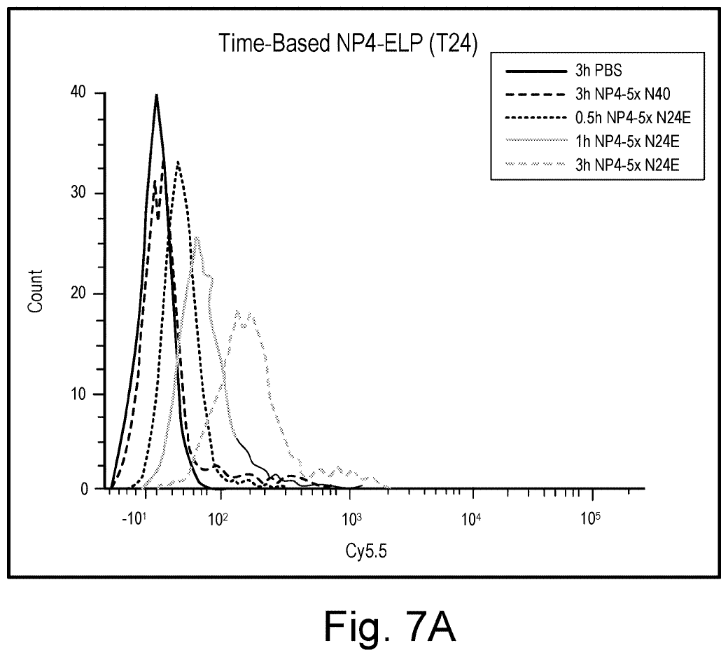

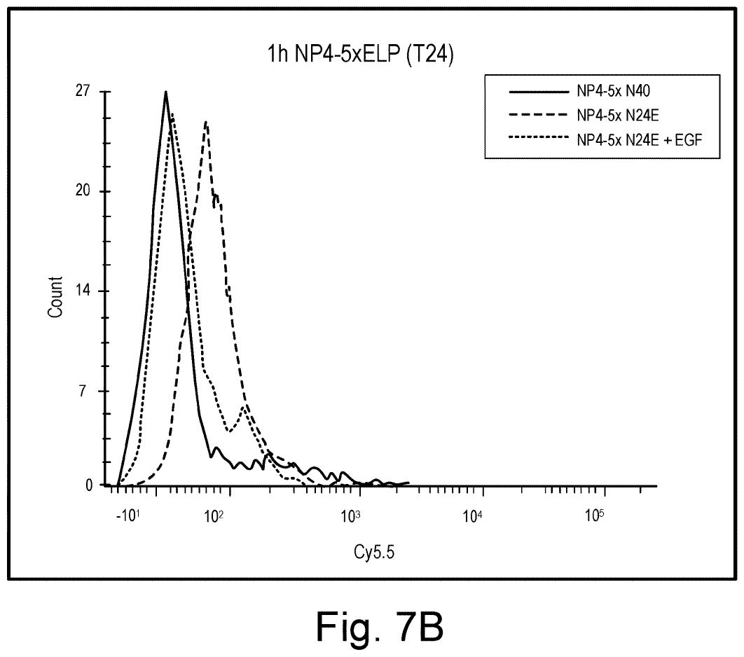

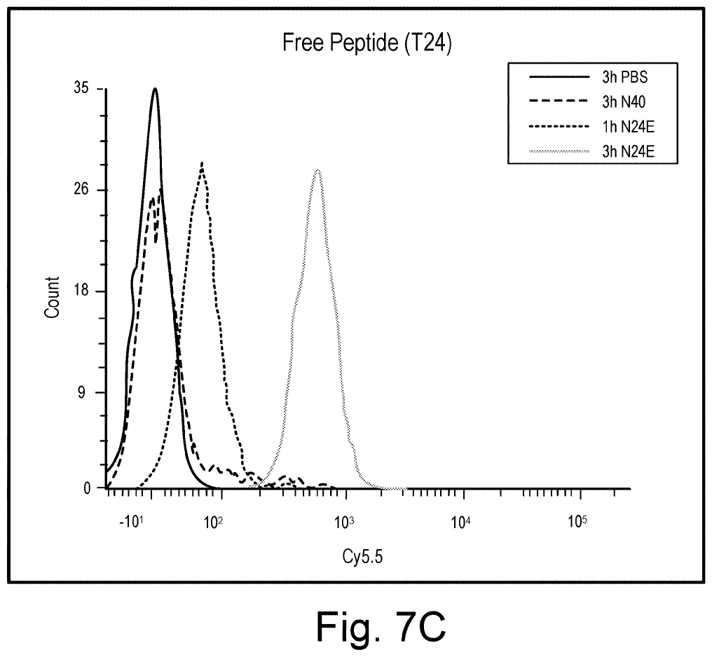

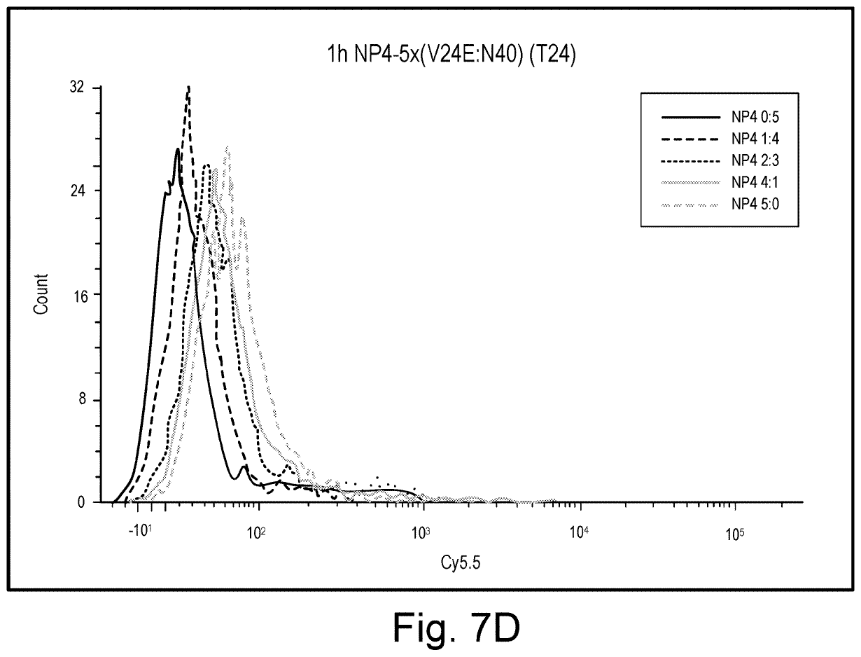

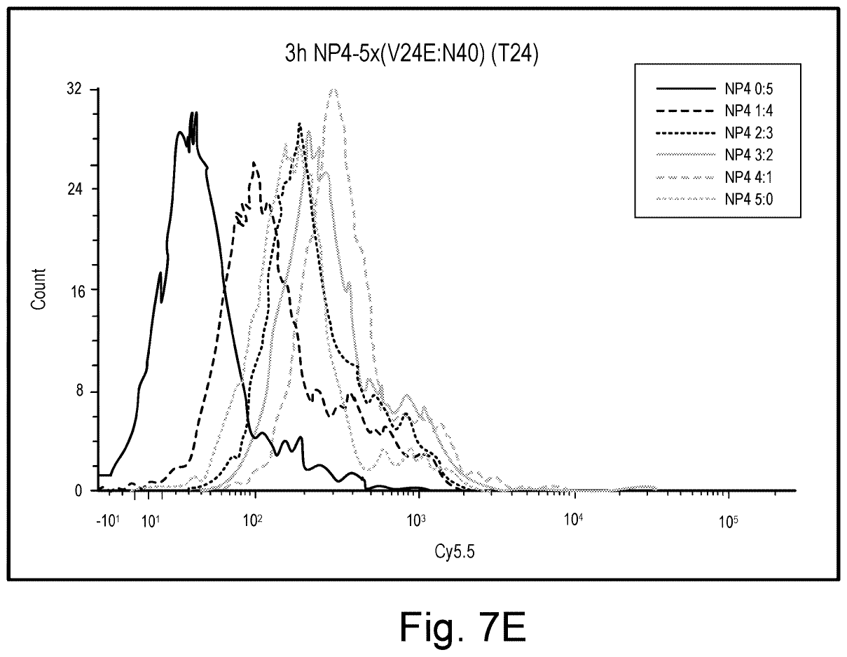

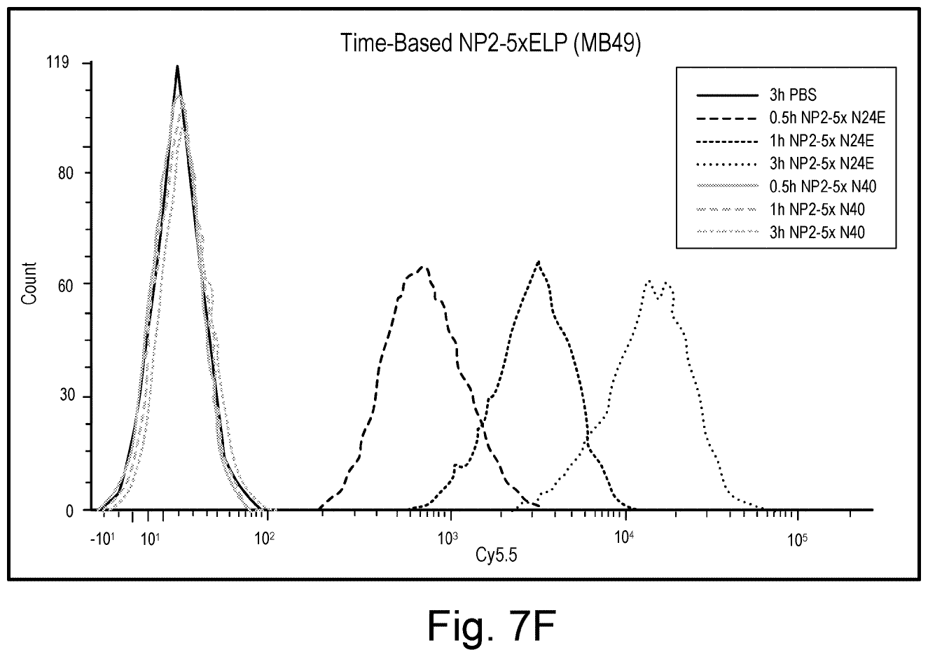

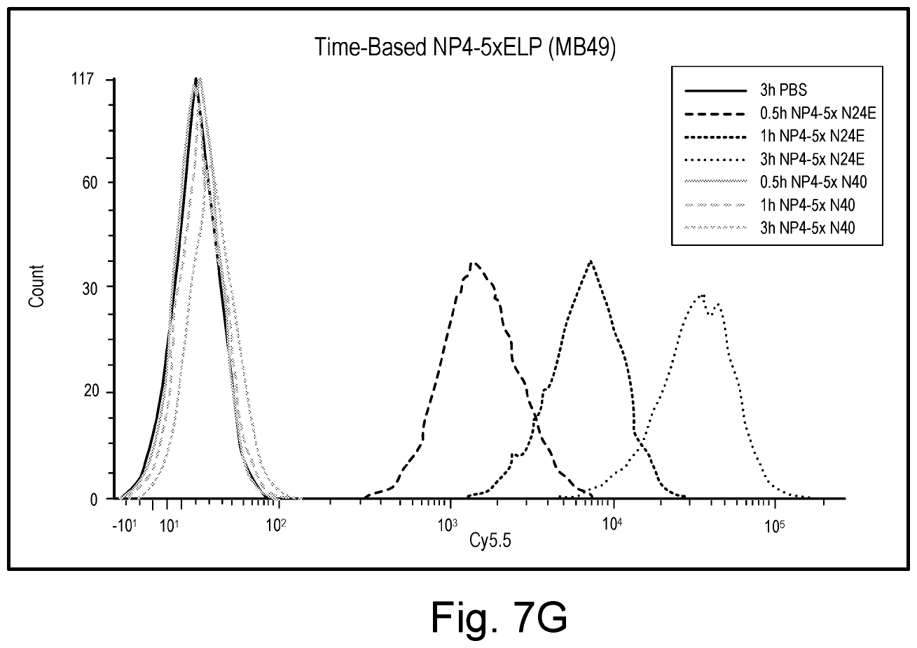

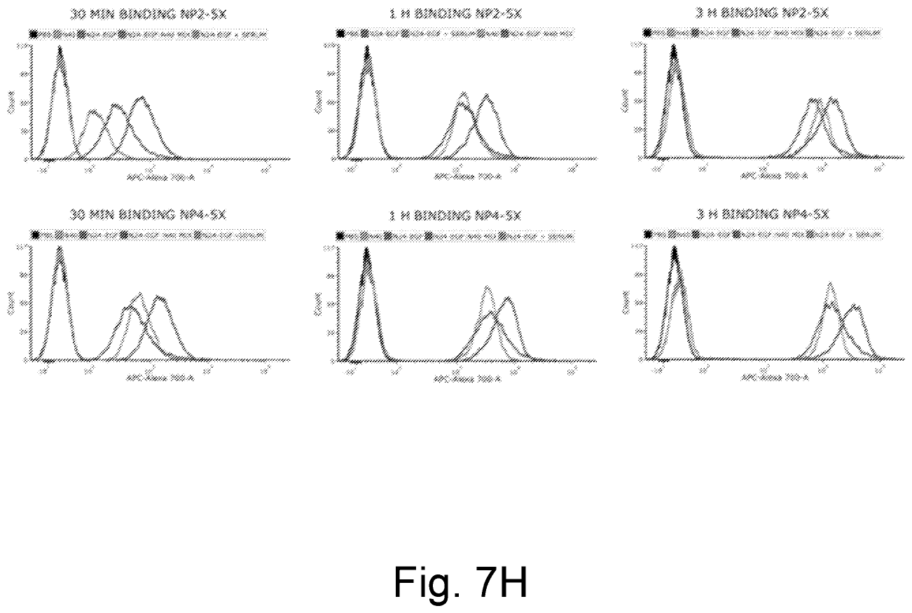

With reference to FIGS. 7A-7E, Cy5.5-conjugated N24-EGF (targeted) and N40 (non-targeted) were used to form siRNA LENN complexes for flow cytometric analysis of their interaction with T24 (human) and MB49 (murine) bladder cancer cells. We observed that Cy5.5-N24-EGF containing LENN produced a strong cell associated fluorescence in both cell lines that increased with incubation time (FIG. 7A, 7F, 7G). Conversely, little to no Cy5.5 fluorescence was observed in T24 cells incubated with Cy5.5N40 LENN at NP4 for up to 5 h. These findings demonstrated that the targeted formulations had enhanced cell association through engagement of EGF receptors (EGFR) on the tumor cell surface. Further support for this conclusion was provided by EGFR blockade studies using serum-supplemented media before treatment with the LENN formulations that produced a reduction in cell-associated fluorescence due to competition for EGFR by LENN and the free EGF present in the media (FIG. 7B). The cell-associated fluorescence stagnates by 2 h in T24, which was attributed to maximum binding and internalization by that time point. Importantly, 2 h is the practical therapeutic window for agents administered by intravesical infusion; wash out through micturition typically occurs beyond this time window. Moreover, MB49 is a higher EGFR-expressing cell line than T24, which accounts for the higher cell-associated fluorescence levels observed upon treatment of MB49 cells with N24EGF containing formulations. Formulations prepared at lower NP ratios, but the same ELP ratio, also showed lower cell-associated fluorescence, likely due to the lability of these complexes that impeded their efficient binding and/or internalization (e.g., T24: NP2-5×N24EGF in FIG. 7H vs. NP4-5×N24EGF in FIG. 7A; MB49: NP2-5×N24-EGF and NP4-5×N24EGF in FIG. 7F vs. FIG. 7G). Free ELP and ELP-wrapped siRNA:CDPLR10 LENN complexes have similar cell-associated fluorescence, at least up to 1 h (FIG. 7C vs. FIGS. 7A and 7B). Serum-based competition had been shown to occur within 30 min when the free N24-EGF peptide was incubated with T24 cells, unlike N24-EGF LENN formulations that display greater avidity for T24 cells in the presence of serum (FIG. 7H). This resistance to serum competition at 30 min was determined to arise from two potential origins: (1) reduced availability of free receptors on the T24 cell surface due to multivalent engagement of EGFR by LENN that present multiple copies of spatially restricted EGF; and/or (2) EGF present in the culture medium may stimulate micropinocytosis, a secondary mechanism of EGFR uptake, leading to the observed initial increase in fluorescence upon addition of serum as occurs for other tumor cell lines.

EGF titrations were then performed to evaluate the relationship between cell interaction efficiencies as a function of N24EGF content within blended LENN formulations (FIGS. 7D and 7E). After 1 h incubation, we observed increased cell-associated fluorescence as the EGF content in the LENN nanoparticles increased from 0-100% N24EGF (FIG. 7D). Interestingly, after 3 h incubation, LENN formulated with 100% N24EGF showed less enhancement in cell-associated fluorescence compared to LENN with lower N24EGF loading (c.f., FIG. 7E), 5:0 vs. 4:1 & 3:2 N24EGF). Since EGFR is internalized primarily through clathrin-mediated endocytosis and recent mechanistic studies on the effect of nanoparticle elasticity on tumor uptake shows that stiff spherical particles are more readily internalized by clathrin-mediated pathways than deformable nanoparticles, the increased cell-associated fluorescence with increasing EGF content suggested that multivalent engagement of EGFR may serve to rigidify the LENN. In that scenario, enhanced uptake and EGFR recycling would be expected until receptor saturation occurs.

### Example 6: Qualitative Confocal Microscopy Analysis of Transfection of T24 Bladder Cancer Cells with siRNA-LENN Complexes

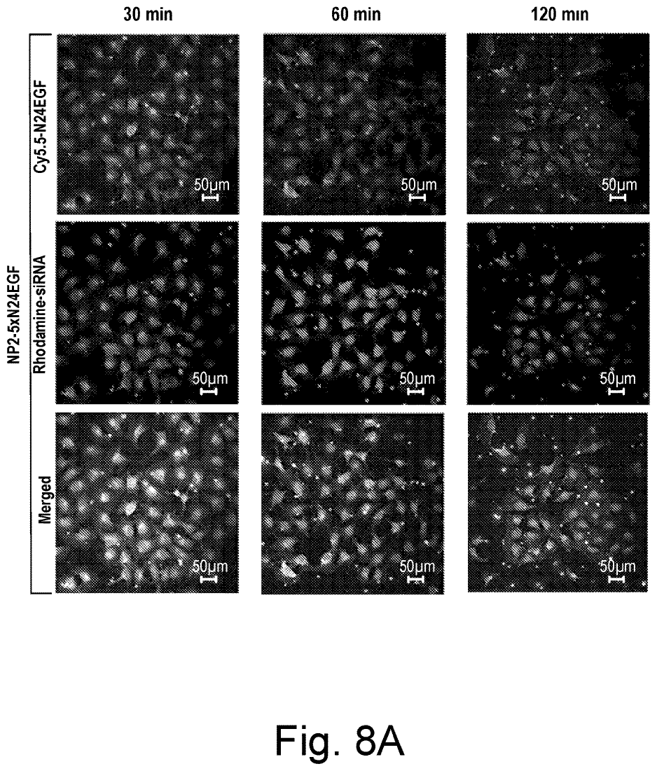

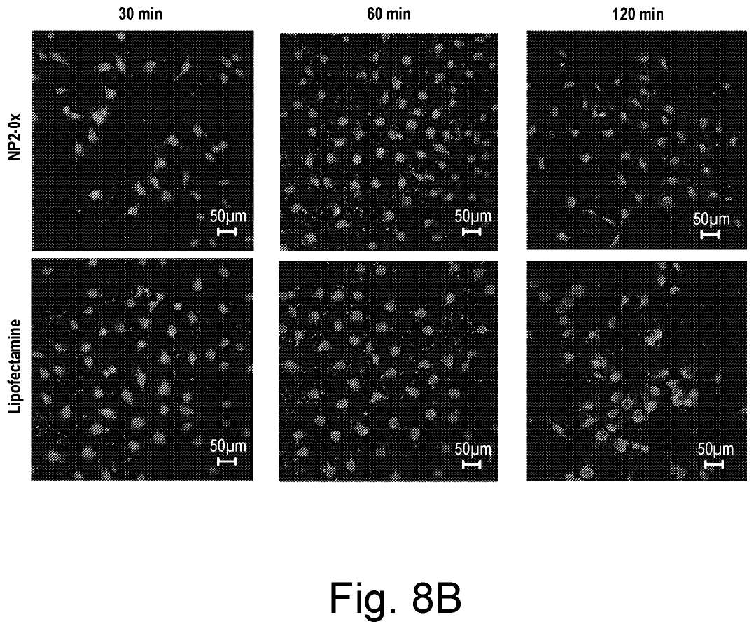

As reproduced in FIG. 8A, confocal microscopy images of T24 cells were collected after incubating them with dual labeled LENN formulated with Cy5.5-labeled ELP and Rhodamine-siRNA to determine the time-dependent distribution of LENN components after binding. Data showed that EGF-targeted LENN are internalized within 30 min and to a greater extent than either Lipofectamine or the polyion complexes lacking an ELP LbL coating. The images also reveal an initial loss of co-localized Cy5.5 and Rh signals within 1 h, an observation interpreted as the onset of LENN dissociation into different cellular compartments. Taken together, these findings showed the general trend of increased dispersion of both the Cy5.5 and Rhodamine signals with increasing incubation time, such that the targeted Cy5.5-N24EGF LENN formulations showed greater uptake and intracellular distribution than polyion complexes at NP=2 or Lipofectamine controls.

### Example 7: Qualitative Confocal Microscopy Analysis of Transfection with siRNA-LENN Complexes

Confocal images reproduced in FIG. 8B were collected after incubating MB49 cells with dual labeled LENN formulated using either Lipofectamine, non-targeted Cy5.5-N40, or targeted Cy5.5-N24EGF to encapsulate FITC-modified pDNA. It was observed that targeted LENN performed better than the commercial transfection standard with respect to tumor association and internalization efficiency. No significant internalization was detected in cells treated with the untargeted LENN, underscoring the importance of EGF in the formulation for engagement and uptake of the LENN vector.

It was also observed that diffuse fluorescence spread over the cells after incubating T24 and MB49 with just Cy5.5-N24EGF, consistent with previous observations in Cy5.5-N24EGF studies. 22 Moreover, the binding kinetics of these N24EGF LENN within the first 30 min appears similar to the free peptide binding rates. From these observations, it was concluded that the cell-association avidity of N24EGF is retained even when present in nanoparticle formulations, thus setting the stage for testing the biological efficacy of the LENN delivery systems in mammalian tumor cells.

### Example 8: SiRNA LENN Mediated Silencing of Hypoxia-Inducible Factor-1α (HIF-1α)

In an effort to evaluate the NA delivery efficiency of the LENN system, HIF-1α siRNA knockdown was used due to the critical role HIF-1α serves in the evolutionary progression of bladder cancer and its dissemination induced by the tumor microenvironment. Several recent studies have revealed the negative impact of hypoxia-inducible factors (HIF) expression in urothelial carcinoma and its positive correlation with chemotherapy resistance, immunotherapy failures, and resistance to BCG therapy. HIF-1α is a transcription factor that is stably expressed in hypoxic environments. Bladder tumors are a prime example of a tissue type that possesses hypoxic microenvironmental niches and limited glucose supply due to poor vascularization. The combination of the two environmental stressors generally leads to aggressive behaviors in tumors through HIF-1α activation of various pro-tumorigenic cellular pathways, ultimately increasing tumor plasticity and functional heterogeneity. This leads to the activation of ‘novel’ metabolic escape pathways, higher invasive capacity, resistance to cytotoxicity, increase in checkpoint inhibitor expression, and other immune suppression pathways.

Overexpression of the RTK membrane protein EGFR has been reported in 74% of bladder cancer patients and has been demonstrated to play a role in many pre- and pro-oncogenic pathways responsible for cell survival, repair, migration, invasion, and cell differentiation. Many therapeutic approaches have been developed to target suppression of EGFR function with mixed success. Many of these have focused on kinase inhibition, however, upon development of tumor resistance towards these therapies, it was discovered that even in the absence of its kinase functions, EGFR participates in pro-survival functions in tumors. From a drug delivery perspective, the higher abundance of EGFR on tumors (high and low grades) compared to normal cells, and its capacity for receptor-mediated endocytosis upon ligand binding, makes it an attractive route for intracellular delivery of nucleic acid loaded LENN bearing EGF targeting ligands.

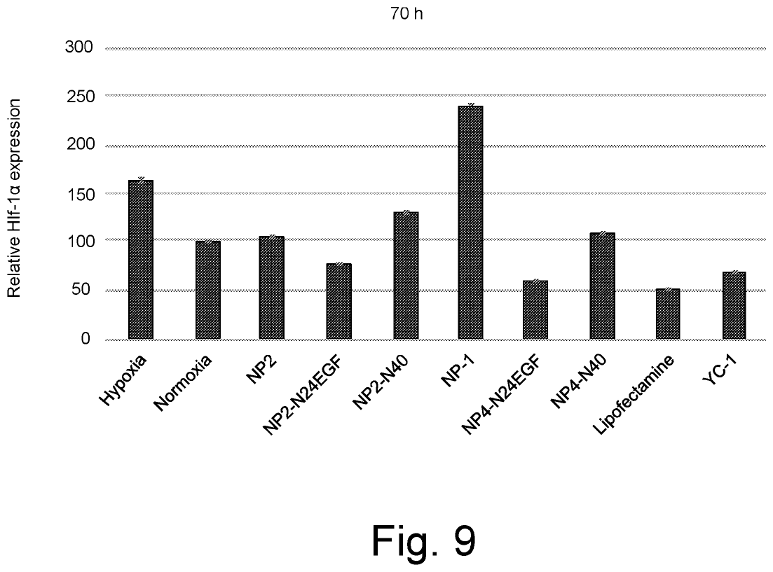

The presence of abrogated HIF-1α mRNA expression was tested using RT-PCR after incubating the cells with various formulations of siRNA:CDPLR₁₀:ELP LENN. In general, as shown in FIG. 9, the targeted siRNA LENN complexes produced greater gene silencing than the non-targeted siRNA LENN or siRNA:CDPLR₁₀ polyion complexes lacking the N24EGF coating. These findings are consistent with an enhanced delivery of functional siRNA to the cytoplasm of bladder tumor cells mediated by EGFR uptake of the EGF-bearing LENN complexes.

### Example 9: Transfection of Murine Bladder Cancer (MB49) and Macrophage (RAW264.7) Cells by pDNA LENN

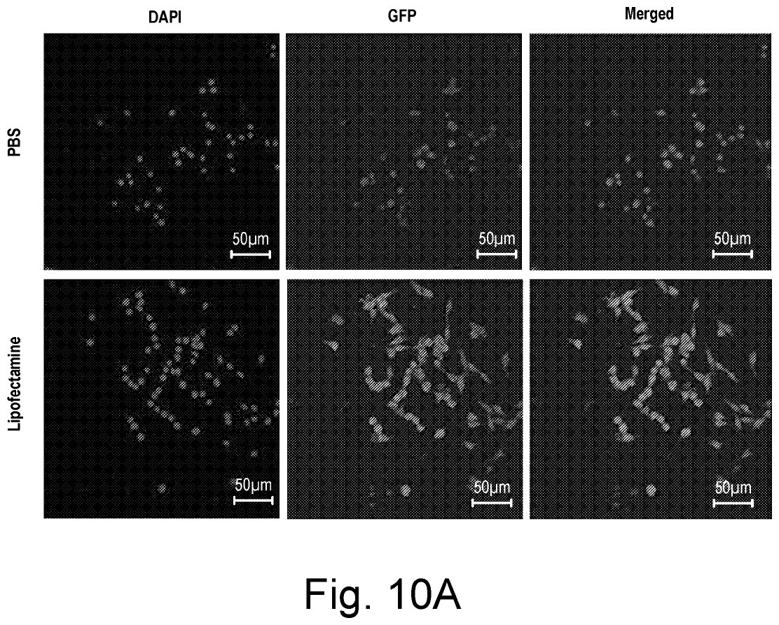

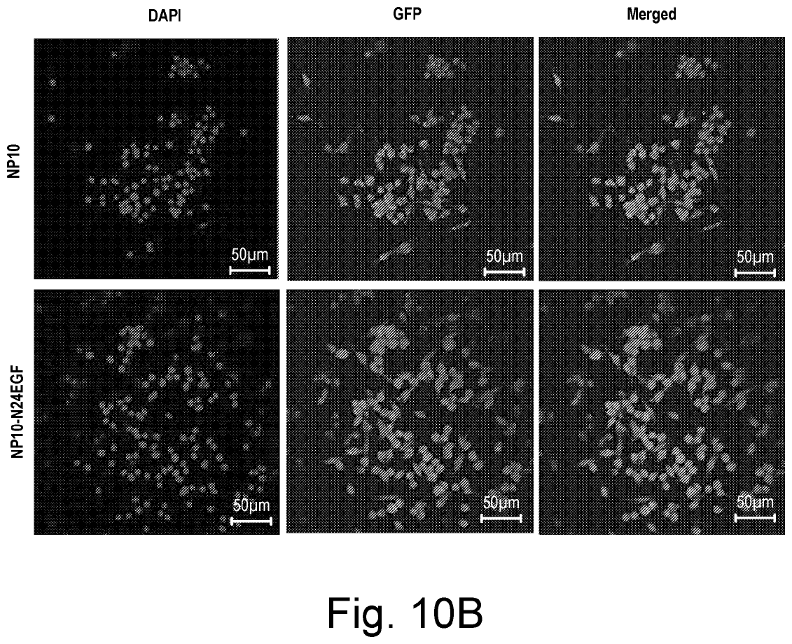

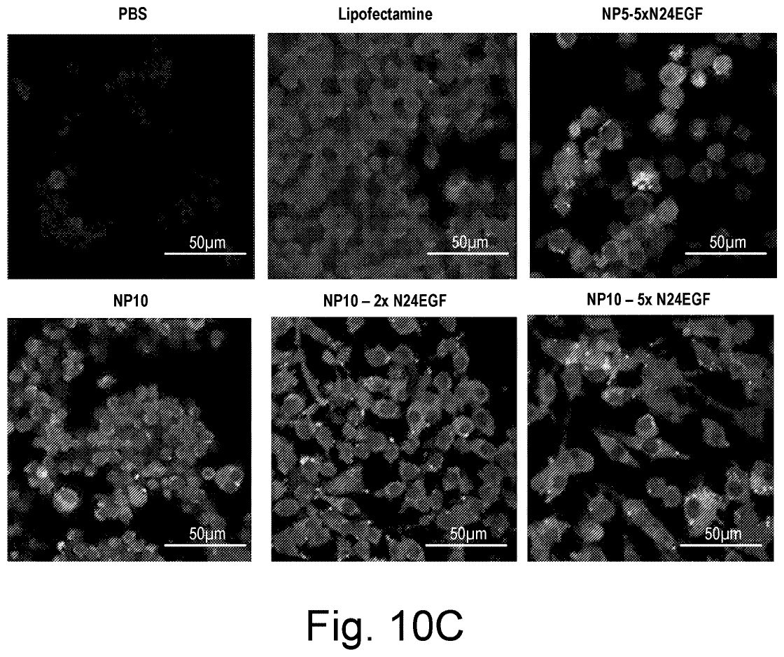

As a further test of the adaptability of the LENN system, delivery of large pDNA payloads in MB49 and RAW264.7 cells was tested for gene expression efficiency. MB49 cells were treated with pDNA:CD-PLR₁₀:ELP LENN and evaluated for GFP expression at 48 h post-transfection Formulations with N24EGF (FIG. 10B) produced maximum GFP expression compared to Lipofectamine and polyion complexes lacking an ELP coating (FIG. 10A). Cytotoxicity was higher in the cases of cells treated with Lipofectamine and polyion complexes as suggested by lower overall cell numbers in those wells (FIG. 10C). High GFP expression in MB49 cells was also observed for pDNA:CDPEI_{2.5k} LENN formulations (FIG. 10B) compared to their polyion complexes lacking an ELP coating or Lipofectamine controls (FIG. 10A).

In bladder cancer, there is generally a close spatial interaction between tumor-associated macrophages and tumors. Our next experiments used RAW264.7 cells to mimic the particle uptake process by tumor-resident macrophages. pDNA:CDPLR₁₀:ELP LENN and pDNA:CDPLR₁₀ complexes were more successful at plasmid transfection than Lipofectamine (FIG. 10C). A similar positive correlation between EGF loading and GFP expression was also observed when CDPEI_{2.5k} was used in the formulations. Although RAW24.7 cells do have a baseline EGFR expression that varies in response to its activation, prior studies showed that up to 5 ng/ml EGF did not lead to enhancement in EGFR expression in RAW264.7. Since the amount used in our formulations is in the sub-100 μg/mL range, the increased GFP expression level was likely attributed to enhanced efficacy rather than stimulated EGFR expression. In any case, the tumor-associated macrophages in bladder cancer are typically M2-like (pro-tumorigenic). Since it has been reported that activation of EGFR in such cases mediates inhibition of M2 polarization, the uptake of EGF-decorated particles by macrophages may result in abrogating pro-tumorigenic tendencies of the macrophages.

In sum, the testing reported herein demonstrates that ELP-based LENN formulations can efficiently deliver short nucleic acid sequence cargo like siRNA as well as ~6 kb constructs like pDNA using the same components in a layer-by-layer assembly process. In addition to the wide range of nucleic acid cargo capacities that this approach affords, it shares the broad compositional variations offered by LNP approaches without their known physical and chemical instability liabilities. Importantly, the formulation precursors used in the LENN complexes are bio-manufacturable on scale, chemically stable, and display low immunogenicity.

In addition to the biomanufacturing advantages provided by this nucleic acid delivery approach, this system is capable of (1) formulation using a layer-by-layer approach to provide high adaptability for engaging the desired biological target using blended ELP components to provide the desired functional properties; (2) cargo release kinetics that are governed by the polycationic ponytail on the cyclodextrin and the nature of the ELP; (3) suppression of off-target engagement due to the steric stabilization properties of ELP; (4) obviation of the known liabilities of bioactive lipid formulation due to uncontrolled phase transitions and chemical inactivation by lipidation of the nucleic acid cargo; and (5) siRNA mediated silencing and pDNA expression using a carrier system with low toxicity.

In the disclosure, the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In the disclosure, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

What is claimed is:

1. A method of preparing an elastin-like polypeptide (ELP) carrier system for delivery of a nucleic acid cargo to over-expressing EGFR (epidermal growth factor receptor) tumor cells, the method comprising:
complexing nucleic acid material of the nucleic acid cargo with decaarginine-β-cyclodextrin (CD) to yield a CD-BP polyplex cargo;
coating the CD-BP polyplex cargo with an ELP-epidermal growth factor (ELP-EGF) fusion protein to yield the ELP carrier system.

2. The method of claim 1, wherein the nucleic acid material comprises one or more of: siRNA, pDNA, miRNA, mRNA, and dsDNA.

3. The method of claim 1, wherein the tumor cells are bladder tumor cells.

4. The method of claim 1 wherein the ELP is a (VPGXG) n construct, wherein n represents the number of pentapeptide repeats and X represents a predetermined amino acid.

5. The method of claim 4, wherein X is valine and n=24.

6. An elastin-like polypeptide (ELP) carrier system for delivery of a nucleic acid cargo to over-expressing EGFR (epidermal growth factor receptor) tumor cells comprising:
a nucleic acid material complexed with decaarginine-β-cyclodextrin (CD); and
an outer coating wrapping, at least in part, the CD-nucleic acid complex with one or more ELP-epidermal growth factor (ELP-EGF) fusion proteins.

7. The ELP carrier system of claim 1, wherein the nucleic acid material comprises one or more of: siRNA, pDNA, miRNA, mRNA, and dsDNA.

8. The ELP carrier system of claim 1, wherein the tumor cells are bladder tumor cells.

9. The ELP carrier system of claim 1, wherein the ELP is a (VPGXG) n construct, wherein n represents the number of pentapeptide repeats and X represents a predetermined amino acid.

10. The ELP carrier system of claim 9, wherein X is valine and n=24.

11. The ELP carrier system of any of claims 6-8 comprising a layer-by-layer ELP-nucleic acid (NA) nanoparticle (LENN) complex.

12. A layer-by-layer ELP-nucleic acid (NA) nanoparticle (LENN) complex comprising:
a polyion complex core wrapped, at least in part, by an ELP fusion protein coating,
wherein the polyion complex core comprises a nucleic acid cargo condensed by an oligocation and the ELP fusion protein comprises an EFP linked directly or indirectly to a co-expressed targeting ligand.

13. The LENN complex of claim 12, wherein the nucleic acid cargo is therapeutic DNA comprising one or more of pDNA, mcDNA, a linear DNA amplicon, an ASO, a CpG oligodeoxynucleotide, and a DNA enzyme.

14. The LENN complex of claim 12, wherein the nucleic acid cargo is therapeutic RNA comprising one or more of mRNA, siRNA, shRNA, miRNA, piRNA, eRNA, saRNA, lncRNA, circRNA, a ribozyme, an antagonist RNA aptamer, and a cell type-specific RNA aptamer.

15. The LENN complex of claim 12, wherein the nucleic acid cargo comprises a CRSPR-Cas9 vector.

16. The LENN complex of claim 12, wherein the oligocation is decaarginine-β-cyclodextrin (CDPLR10) or 2.5 kD polyethyleneimine-modified β-cyclodextrin (CDPEI_{2.5k}).

17. The LENN complex of claim 12, wherein the targeting ligand is a peptide or protein capable of selective interaction with a targeted membrane protein or glycoprotein implicated in cancer behavior the expression of which may be characterized as either (1) overexpressed (or upregulated) in bladder tumors compared to normal tissues or (2) aberrantly expressed in bladder tumors but not in normal tissues.

18. The LENN complex of claim 17, wherein the targeting ligand is a peptide or protein capable of selective interaction with an upregulated integrin, receptor tyrosine kinase (RTK), or G-protein coupled receptors.

19. The LENN complex of claim 17, wherein the targeting ligand is a peptide or protein capable of selective interaction with one of αEβ7 integrin, integrin α2β1, integrin β8, EGFR, FGFR, HER 2, VEGFR-2, or chemokine receptor 7 (CCR7).

20. The LENN complex of claim 17, wherein the membrane protein is EGFR and the ELP fusion protein is N24EGF.

21. The LENN complex of claim 17, wherein the targeting ligand is a peptide or protein capable of selective interaction with an aberrantly expressed cell surface adhesion molecules (CAMs).

22. The LENN complex of claim 21, wherein the targeting ligand is a peptide or protein capable of selective interaction with NECTIN-4 or the surface adhesion glycoprotein TROP-2.

23. A method of formulating a LENN complex of any one of claims 12-22 using a layer-by-layer deposition process (LbL), wherein the method comprises:
condensing a nucleic acid material with a β-cyclodextrin oligocation to form a polyion complex; and
wrapping at least a portion of the polyion complex with an ELP fusion protein to form the LENN complex.

24. The method of claim 23, wherein the LENN complex is prepared with a 5-fold molar excess of prolines plus valines of hydrophobic prosthetic groups of the ELP with respect to cavities of the β-CD and with N:P at a value from 2 to 5.

25. A method of treating bladder cancer in a subject via LENN-mediated delivery of a therapeutic NA cargo, the method comprising administering to the subject a therapeutically effective amount of a composition of LENN complex along with one or more pharmaceutically acceptable carriers,
wherein the administering affects intracellular delivery of the condensed NA cargo to bladder tumor cells.

26. The method of claim 25, wherein the administering is performed via intravesical infusion.
